(19) United States
(12) Patent Application Publication
(10) Pub. No.: US 2026/0260738 A1
(43) Pub. Date: Sep. 3, 2026

(54) COLLABORATIVE INTERCONNECTED RESPONSE AND COORDINATION TECHNOLOGIES

(71) Applicant: InSight Collaborative Solutions LLC, Delaware, OH (US)

(72) Inventors: Lorelle Scheuer, Delaware, OH (US); Madeline Stern, Westlake, OH (US)

(21) Appl. No.: 19/445,913

(22) Filed: Jan. 12, 2026

Related U.S. Application Data
(60) Provisional application No. 63/743,738, filed on Jan. 10, 2025.
(60) Provisional application No. 63/905,110, filed on Oct. 24, 2025.

(51) Int. Cl. G16H 40/20

(52) U.S. Cl. CPC G16H 40/20

(57) ABSTRACT

Technologies for enhanced issue tracking and resolution include a computing system configured to receive a request to present a form associated with an issue to be resolved by an organization. The request is associated with a user that is associated with a corresponding tier of the organization. The computing system is further configured to present a form to represent issue data indicative of an issue to be resolved by the organization and store the issue data represented in the form, in association with a version of the form for the tier corresponding with the user. At least a portion of the issue data is stored to be prepopulated into a different version of the form for a different tier of the organization.

### CROSS-REFERENCE TO RELATED APPLICATIONS

This application claims priority to and the benefit of U.S. Provisional Application No. 63/743,738, titled “Collaborate Interact Resolve Communicate Form,” filed on Jan. 10, 2025, and U.S. Provisional Application No. 63/905,110, titled “Collaborate Interact Resolve Communicate Form,” filed on Oct. 24, 2025, the contents of which are incorporated herein by reference in their entirety.

### BACKGROUND

Tracking and resolving issues within an organization is inefficient due to technical shortcomings of conventional issue tracking systems. While conventional systems may provide a surface-level sense of collaboration through shared access and manual editing, they quickly prove to be ineffective when applied to complex, multi-tiered workflows. Some conventional systems, for example, may route incident, request, problem, and change forms through siloed organizational structures. Updates in such systems are typically segmented by role, and visibility is gated by user assignment. Accordingly, conventional systems lack the technical features required to provide transparency, adaptability, and real-time coordination required for true cross-functional engagement within an organization. These technical shortcomings of conventional systems may allow issues that adversely affect the efficiency of an organization to remain unresolved, potentially leading to excessive consumption of time, energy, or other resources that could otherwise be avoided.

### SUMMARY

One embodiment is directed to a unique system, components, and methods for enabling enhanced issue tracking and resolution for an organization. Other embodiments are directed to apparatuses, systems, devices, hardware, methods, and combinations thereof for enabling enhanced issue tracking and resolution for an organization.

According to an aspect of the present disclosure, a method for enhanced issue tracking and resolution includes receiving, by a computing system, a request to present a form associated with an issue to be resolved by an organization. The request is associated with a user that is associated with a corresponding tier of the organization. The method also includes presenting, by the computing system, a form to represent issue data indicative of an issue to be resolved by the organization. Further, the method includes storing, by the computing system, the issue data represented in the form, in association with a version of the form for the tier corresponding with the user. At least a portion of the issue data is stored to be prepopulated into a different version of the form for a different tier of the organization. The organization may be a health care organization in which medical services are provided to patients by personnel of the organization. The personnel may include nurses and issues may be resolved in connection with nursing huddles (“huddles”) in which personnel of the health care organization may share status updates and information regarding medical services to be provided for patients. As such, through enhanced tracking and resolution of issues that affect the ability of the health care organization to provide the medical services, the method enables the health care organization to more efficiently perform medical services for patients.

In some embodiments, the method also includes determining by an issue routing system, whether the issue data warrants escalation or de-escalation of the issue and selectively escalating or de-escalating, by the computing system, the issue as a function of the determination of whether the issue data warrants escalation or de-escalation of the issue.

In some embodiments, presenting the form to represent issue data comprises presenting a form with a set of fields to each represent a corresponding portion of the issue data.

In some embodiments, presenting the form comprises presenting a version of the form with fields associated with the tier of the user.

In some embodiments, presenting the form comprises presenting a form that is associated with an existing issue associated with the organization.

In some embodiments, the method further includes retrieving data to pre-populate fields of the form as a function of issue data stored in association with at least one version of the form associated with a tier other than the tier of the user.

In some embodiments, the method further includes prompting the user to enter portions of the issue data as a function of workflow logic defined in association with the form, such that the version of the form for the tier of the health care organization corresponding with the user remains independent from the one or more other versions of the form associated with the one or more other tiers, and issue data is selectively propagated between versions of forms by the computing system, wherein presenting the form comprises presenting, to the user, communications associated with the form and designated for distribution to the user based on a distribution group, and wherein presenting the form further comprises constraining user input to the form such that the issue data is categorized, machine-readable, and comparable to issue data in other forms.

In some embodiments, the method further includes presenting bottleneck data indicative of a number of other issues having a common due date with the issue associated with the form.

In some embodiments, the method further includes receiving an adjusted due date based on the presented bottleneck data and presenting recalculated bottleneck data as a function of the adjusted due date.

In some embodiments, storing the issue data represented in the form comprises overwriting prepopulated data from one or more other versions of the form associated with one or more other tiers with data entered by the user into the corresponding fields.

In some embodiments, the method further includes determining, by an issue routing system, whether the issue data warrants escalation or de-escalation of the issue, selectively escalating, by the computing system, the issue as a function of the determination of whether the issue data warrants escalation or de-escalation of the issue, and providing, by the computing system, a notification to a target user associated with a higher tier than the tier of the user to review the issue data in a corresponding version of the form for the tier of the target user.

In some embodiments, the method further includes determining, by an issue routing system, whether the issue data warrants escalation or de-escalation of the issue, selectively escalating, by the computing system, the issue as a function of the determination of whether the issue data warrants escalation or de-escalation of the issue, and providing a notification to a target user associated with a lower tier than the tier of the user to review the issue data in a corresponding version of the form for the tier of the target user.

In some embodiments, the method further includes displaying a workboard that represents a set of issues to be resolved by the organization and that are associated with the user.

In some embodiments, the method further includes displaying a summary of issue data associated with each issue of the set of issues represented by the workboard.

In some embodiments, the method further includes displaying a status of each issue of the set of issues represented by the workboard.

According to another aspect of the present disclosure, a computing system for enhanced issue tracking and resolution includes at least one processor and at least one memory comprising a plurality of instructions stored thereon that, in response to execution by the at least one processor, causes the computing system to receive a request to present a form associated with an issue to be resolved by an organization. The request is associated with a user that is associated with a corresponding tier of the organization. The instructions further cause the computing system to present a form to represent issue data indicative of an issue to be resolved by the organization and store the issue data represented in the form, in association with a version of the form for the tier corresponding with the user. At least a portion of the issue data is stored to be prepopulated into a different version of the form for a different tier of the organization.

In some embodiments, the instructions additionally cause the computing system to determine by an issue routing system, whether the issue data warrants escalation or de-escalation of the issue and selectively escalate or de-escalate the issue as a function of the determination of whether the issue data warrants escalation or de-escalation of the issue.

In some embodiments, to present the form to represent issue data comprises to present a form with a set of fields to each represent a corresponding portion of the issue data.

In some embodiments, to present the form comprises to present a version of the form with fields associated with the tier of the user.

In some embodiments, to present the form comprises to present a form that is associated with an existing issue associated with the organization.

In some embodiments, the instructions additionally cause the computing system to retrieve data to pre-populate fields of the form as a function of issue data stored in association with at least one version of the form associated with a tier other than the tier of the user.

In some embodiments, the instructions additionally cause the computing system to prompt the user to enter portions of the issue data as a function of workflow logic defined in association with the form.

In some embodiments, the instructions additionally cause the computing system to present bottleneck data indicative of a number of other issues having a common due date with the issue associated with the form.

In some embodiments, the instructions additionally cause the computing system to receive an adjusted due date based on the presented bottleneck data and present recalculated bottleneck data as a function of the adjusted due date.

In some embodiments, to store the issue data represented in the form comprises to overwrite prepopulated data from one or more other versions of the form associated with one or more other tiers with data entered by the user into the corresponding fields.

In some embodiments, the instructions additionally cause the computing system to determine, by an issue routing system, whether the issue data warrants escalation or de-escalation of the issue, selectively escalate the issue as a function of the determination of whether the issue data warrants escalation or de-escalation of the issue, and provide a notification to a target user associated with a higher tier than the tier of the user to review the issue data in a corresponding version of the form for the tier of the target user.

In some embodiments, the instructions additionally cause the computing system to determine, by an issue routing system, whether the issue data warrants escalation or de-escalation of the issue, selectively escalate the issue as a function of the determination of whether the issue data warrants escalation or de-escalation of the issue, and provide a notification to a target user associated with a lower tier than the tier of the user to review the issue data in a corresponding version of the form for the tier of the target user.

In some embodiments, the instructions additionally cause the computing system to display a workboard that represents a set of issues to be resolved by the organization and that are associated with the user.

In some embodiments, the instructions additionally cause the computing system to display a summary of issue data associated with each issue of the set of issues represented by the workboard.

In some embodiments, the instructions additionally cause the computing system to display a status of each issue of the set of issues represented by the workboard.

This summary is not intended to identify key or essential features of the claimed subject matter, nor is it intended to be used as an aid in limiting the scope of the claimed subject matter. Further embodiments, forms, features, and aspects of the present application shall become apparent from the description and figures provided herewith.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 depicts a simplified block diagram of at least one embodiment of a system for enabling enhanced issue tracking and resolution for an organization;

FIG. 2 is a simplified block diagram of at least one embodiment of a computing device;

FIGS. 3-5 are a simplified flow diagram of at least one embodiment of a method for enabling enhanced issue tracking and resolution for an organization that may be executed by the system of FIG. 1;

FIGS. 6-8 represent flows of operations that may be executed in connection with the system of FIG. 1;

FIG. 9 represents diagrams of forms that may be produced by the system of FIG. 1;

FIG. 10 represents diagrams of components of workboards that may be produced by the system of FIG. 1; and

FIGS. 11-19 represent user interfaces that may be produced by the system of FIG. 1.

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Further, particular features, structures, or characteristics may be combined in any suitable combinations and/or sub-combinations in various embodiments.

Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

The disclosed embodiments may, in some cases, be implemented in hardware, firmware, software, or a combination thereof. The disclosed embodiments may also be implemented as instructions carried by or stored on one or more transitory or non-transitory machine-readable (e.g., computer-readable) storage media, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

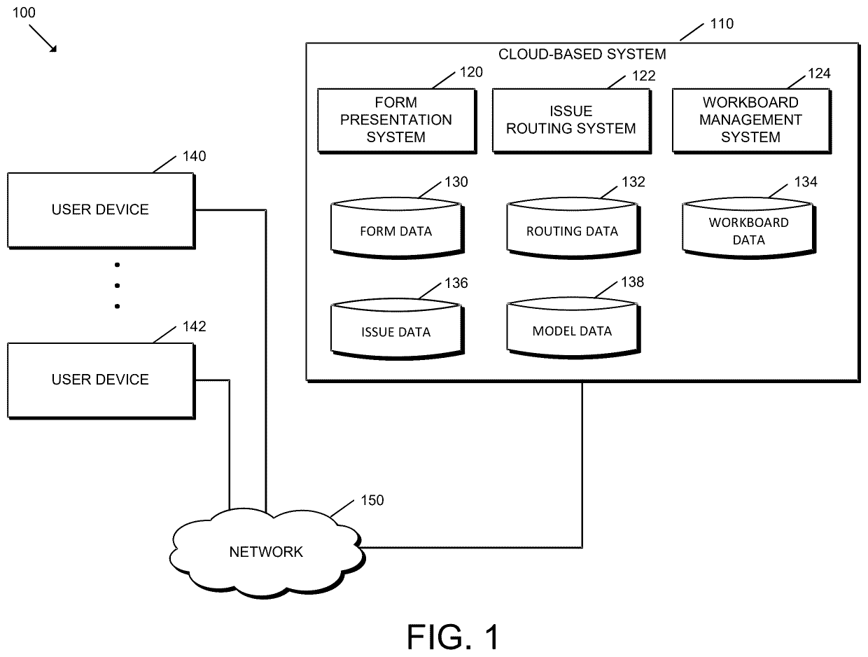

Referring now to FIG. 1, a system 100 (a computing system) for enabling enhanced issue tracking and resolution for an organization includes a cloud-based system 110, a set of user devices 140, 142, and a network 150. The illustrative cloud-based system 110 includes a form presentation system 120, an issue routing system 122, a workboard management system 124, form data 130, routing data 132, workboard data 134, issue data 136, and model data 138. Although only one form presentation system 120, one issue routing system 122, one workboard management system 124, two user devices 140, 142, and one network 150 are shown in the illustrative embodiment of FIG. 1, the system 100 may include any number of form presentation systems 120, issue routing systems 122, workboard management systems 124, user devices 140, 142, and networks 150. For example, in some embodiments, multiple cloud-based systems 110 (e.g., related or unrelated systems) may be used to perform the various functions described herein. Further, in some embodiments, one or more of the systems described herein may be excluded from the system 100, one or more of the systems described as being independent may form a portion of another system, and/or one or more of the systems described as forming a portion of another system may be independent.

The cloud-based system 110 may be embodied as any one or more types of devices/systems capable of performing the functions described herein. For example, as described herein, the form presentation system 120, in the illustrative embodiment, is configured to present an interactive form that prompts a user (e.g., operating a user device 140, 142) and associated with a tier of an organization to provide issue data indicative of an issue to be resolved by the organization. The form presentation system 120 may render a version of a form with a set of user interface elements that correspond to fields or subsections of the issue data 136 associated with the issue to be defined in connection with the form. In doing so, the form presentation system 120 may reference the form data 130 which may define a format for a version of the form and logic to be utilized in interactively presenting the form to the user, such as fields to be selectively hidden or displayed based on information provided by the user in one or more other fields of the form. Further, and as described in more detail herein, the form presentation system 120 presents a version of the form that is associated with a tier of the user viewing the form. Accordingly, the form data 130 may define the format and logic for each of multiple versions of a form, each corresponding to a different tier of the organization. As described in more detail herein, in presenting a form, the form presentation system 120 may prepopulate one or more fields of a version of a form with issue data 136 that was entered into fields associated with a different version of the form associated with a different tier of the organization. Accordingly, for a user in a given tier, that user may see not only fields of issue data 136 pertaining to the tier of that user, but may also see issue data 136 associated with other tiers of the organization in connection with the issue, such as issue data that was provided by user(s) associated with one or more tiers below the tier of the present user. In some embodiments, the form presentation system 120 may present, in a given version of a form, issue data 136 associated with the present issue that was provided by a user from a higher tier (via the version of the form associated with that higher tier).

The issue routing system 122, in the illustrative embodiments, determines whether information entered by a user into a form, presented by the form presentation system 120, warrants escalation or de-escalation of the underlying issue to another tier of the organization. For example, the issue routing system 122 may read a set of routing data 132 which may define rules indicative of conditions under which an issue, as defined by issue data 136 received by the system 110 via a form, should be escalated or de-escalated (e.g., deemed to be resolved). The conditions may be defined as a function of (e.g., based on) the presence of one or more key words indicated in one or more fields, degrees of severity indicated by one or more fields (e.g., a numeric value associated with a given range, words that map to corresponding degrees of severity, etc.), a composite score based on a combination of fields of issue data 136 associated with a form, and/or other factors. Certain embodiments may allow forms to be configured to escalate or de-escalate automatically based on rules of a form, based solely on user discretion, or by prompting the user with a recommendation based on the rules of a form while ultimately allowing the user to decide.

The workboard management system 124, in the illustrative embodiment, produces workboards which may be embodied as user interfaces that indicate the status of issues represented in the issue data 136. Each issue is associated with a corresponding form. In the illustrative embodiment, the workboard management system 124 produces workboards that are defined in accordance with one or more characteristics of a user viewing the workboard. For example, for a given user, the workboard management system 124 provides one or more workboards that represent issues that have been assigned to or are otherwise associated with that user. In the illustrative embodiment, the workboard management system 124 may produce four types of workboards. Those four types may include an active workboard, which may present properties of a submission of an issue, such as date of submission, name of the user who submitted the issue, unit or department associated with the issue, and the type of submission (e.g., type of issue). Additionally, the active workboard may include work notes that provide an overview of information entered on the version of the form associated with the tier of the user that is viewing the workboard. Another type of workboard that may be provided by the workboard management system 124 is a continue to track workboard, which may display a submission description and work notes (e.g., a current owner associated with the issue and overview of information entered by the current owner) for each issue. Additionally, the workboard management system 124 may provide a queue workboard, which may display a relatively brief overview of notes and a target date (e.g., due date) for review and entry of issue data into fields of the corresponding version of the form for the present tier. The workboard management system 124 may also produce a resolved workboard, which includes a submission description and work notes for issues that have been resolved by a tier of the organization. The work notes on the resolved workboard may include an identifier of the tier that resolved the issue, an overview of information provided by the resolving tier, and abbreviated notes from all tiers involved with the issue. Each issue represented in a workboard is associated with a corresponding form, and in the illustrative embodiment, each issue represented on a workboard (e.g., in a row of a table) is represented with a link that, when selected by a user, causes the form presentation system 120 to present the corresponding version of the form for that issue. In the illustrative embodiment, the workboard managements system 124 displays the workboards based on workboard data 134, which may be embodied as a set of data that defines the format and sets of data to be displayed in connection with each issue for each type of workboard.

In at least some embodiments, one or more operations of the cloud-based system 110 may be performed or controlled by an artificial intelligence model, defined in the model data 138. For example, operations associated with the presentation of a form (e.g., for defining the fields and selectively displaying or hiding fields) and/or determining whether to escalate or de-escalate (e.g., deem to be resolved) an issue may be executed or controlled by an artificial intelligence model, which may operate stochastically rather than based on defined rules, and may learn and adjust future operations or determinations based on results of previous operations. Some embodiments may employ one or more artificial intelligence models to, for example, guide users, populate fields, provide coaching on decision-making, suggest next steps, suggest or execute escalation, and/or identify patterns in free-text data. Other functionality may also be provided by artificial intelligence models. Artificial intelligence models may support user decision making by guiding users on whether to escalate and/or suggesting next steps for a given issue. Artificial intelligence models may automatically populate fields (e.g., action owner, Point of Contact, target date, next steps, etc.) based on audio from meetings or huddles. Artificial intelligence models may draft and/or revise communications, data entries in forms, etc. Artificial intelligence models may collect and/or analyze data, for example by scanning free-text entries to identify themes and/or trends, and/or by reviewing data from interfaced systems to identify concerns and/or generate tickets.

One or more of the artificial intelligence models defined in the model data 138 may be a machine learning model. It should be appreciated that a machine learning model refers to an algorithm or collection of algorithms that takes structured and/or unstructured data inputs and generates a prediction or result. The prediction is typically a value or set of values. A machine learning model may itself include one or more component models that interact to yield a result. As used herein, a machine learning model represents both machine learning processing and the model that is created through successive executions of the model. Typically, a model is executed successively during a training phase and after it has been successfully trained, is used operationally to evaluate new data and make predictions. It must be emphasized that the training phase may be executed thousands of times in order to obtain an acceptable model capable of predicting success metrics. Further, the model may discover thousands or even tens of thousands of features. Many of these features may be quite different than the features provided as input data. Thus, the model is not known in advance and the calculations cannot be made through mental effort alone. In some embodiments, the machine learning model/algorithm may utilize one or more neural network algorithms, regression algorithms, instance-based algorithms, regularization algorithms, decision tree algorithms, Bayesian algorithms, clustering algorithms, association rule learning algorithms, deep learning algorithms, dimensionality reduction algorithms, and/or other suitable machine learning algorithms, techniques, and/or mechanisms. It should also be understood, that in some embodiments, the machine learning model may be embodied as a single machine learning model, whereas in other embodiments, the machine learning model may be embodied as a set (ensemble) of machine learning models realizing as a whole the intended function.

In some embodiments, a machine learning model utilized by the system 100 may be a gradient boosted model, which may be embodied as an ensemble of weak learners (e.g., decision trees) that, together, form a more accurate predictive model. The gradient boosted model may be trained with an ensemble metaheuristic to produce the ensemble of weak learners. In other embodiments, the machine learning model may have a different architecture, such as a neural network. A neural network, also referred to herein as an artificial neural network, is a set of connected units or nodes that model the neurons in a brain and that are connected via edges, which model synapses in the brain. Each neuron is configured to receive corresponding signals from connected neurons, then process those signals and produce a resulting signal to other connected neurons. The resulting signal is produced based on an activation function, which is a function that determines an output of a node based on the individual inputs and weights associated with those inputs. In other words, each neuron of an intermediate or last layer may receive an input signal, e.g., a weighted sum of output signals from other neurons, and may process the input signal using a linear or nonlinear function (e.g., an activation function). The activation function may be, for example, a rectified linear unit activation function, a gaussian error linear unit activation function, or a logistic sigmoid function. It should be appreciated that one or more processors (e.g., CPUs or GPUs) or other hardware devices may be dedicated to executing the neural network or other machine learning model in some embodiments. In some embodiments, the system 100 may utilize a recurrent neural network (RNN), which is a specialized type of artificial neural network designed for sequential data processing and that utilizes a recurrent unit that maintains a hidden state that is updated for each of multiple time steps based on a present input and a previous hidden state. A feedback loop may enable the RNN to learn from previous inputs and incorporate that information into the current processing.

Further, for natural language processing operations, the system 100 may use a language model, such as a large language model (LLM). An LLM is a machine learning model designed for natural language processing operations and is trained using self-supervised learning on a relatively large amount of text. The LLM may, in some embodiments, be a generative pretrained transformer (GPT). In a transformer architecture, text is converted into a vector structure through a word embedding table, and in each of multiple layers of the architecture, the transformer contextualizes the token within the scope of a context window with other tokens through a parallel multi-head attention mechanism. Through the architecture, a signal for a key (e.g., significant) token may be amplified and the signal for a less significant token may be de-emphasized. A GPT is a type of generative artificial intelligence framework based on a transformer deep learning architecture that is pre-trained on a relatively large data set of unlabeled text to produce human-like outputs.

Although the cloud-based system 110 is described herein in the singular, it should be appreciated that the cloud-based system 110 may be embodied as or include multiple servers/systems in some embodiments. Further, although the cloud-based system 110 is described herein as a cloud-based system, it should be appreciated that the system 110 may be embodied as one or more servers/systems residing outside of a cloud computing environment in other embodiments. In cloud-based embodiments, the cloud-based system 110 may be embodied as a server-ambiguous computing solution similar to that described below.

Each of the user devices 140, 142 may be embodied as any type of device or system capable of interacting with the cloud-based system 110 (e.g., via a network, using one or more corresponding communication protocols, application programming interface (API) calls, etc.) and/or otherwise capable of performing the functions described herein. In at least some embodiments, the user devices 140, 142 are computer systems associated with one or more organization (e.g., companies) that perform operations to satisfy one or more goals.

The network 150 may be embodied as any one or more types of communication networks that are capable of facilitating communication between the various devices communicatively connected via the network 150 (e.g., the cloud-system 110 and the user devices 140, 142). As such, the network 150 may include one or more networks, routers, switches, access points, hubs, computers, and/or other intervening network devices. For example, the network 150 may be embodied as or otherwise include one or more cellular networks, telephone networks, local or wide area networks, publicly available global networks (e.g., the Internet), ad hoc networks, short-range communication links, or a combination thereof. In some embodiments, the network 150 may include a circuit-switched voice or data network, a packet-switched voice or data network, and/or any other network able to carry voice and/or data. In particular, in some embodiments, the network 150 may include Internet Protocol (IP)-based and/or asynchronous transfer mode (ATM)-based networks. In some embodiments, the network 150 may handle voice traffic (e.g., via a Voice over IP (VOIP) network), web traffic (e.g., such as hypertext transfer protocol (HTTP) traffic and hypertext markup language (HTML) traffic), and/or other network traffic depending on the particular embodiment and/or devices of the system 100 in communication with one another. In various embodiments, the network 150 may include analog or digital wired and wireless networks. For example, the network 160 may include an IEEE 802.11 network, Public Switched Telephone Network (PSTN), Integrated Services Digital Network (ISDN), Digital Subscriber Line (xDSL) network, mobile telecommunications network, wired Ethernet network, private network (e.g., such as an intranet), radio, television, cable, satellite, and/or any other delivery or tunneling mechanism for carrying data, or any appropriate combination of such networks. The network 150 may enable connections between the various devices/systems 110, 120, 122, 124, 140, 142, of the system 100. It should be appreciated that the various devices/systems 110, 120, 122, 124, 140, 142 may communicate with one another via different networks 150 depending on the source and/or destination devices/systems 110, 120, 122, 124, 140, 142.

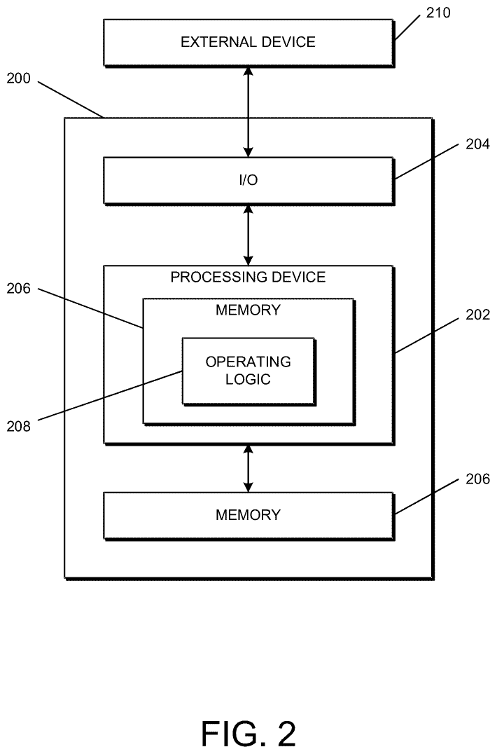

It should be appreciated that each of the cloud-based system 110, the systems 120, 122, 124, the data sets 130, 132, 134, 136, 138, and the user devices 140, 142 may be embodied as, executed by, form a portion of, or associated with any type of device/system, collection of devices/systems, and/or portion(s) thereof suitable for performing the functions described herein (e.g., the computing device 200 of FIG. 2).

Referring now to FIG. 2, a simplified block diagram of at least one embodiment of a computing device 200 is shown. The illustrative computing device 200 depicts at least one embodiment of each of the computing devices, systems, servicers, controllers, switches, gateways, engines, modules, and/or computing components described herein (e.g., which collectively may be referred to interchangeably as computing devices, servers, or systems for brevity of the description). In some embodiments, the computing device 200 may be embodied as a server, desktop computer, laptop computer, tablet computer, notebook, netbook, Ultrabook™, cellular phone, mobile computing device, smartphone, wearable computing device, personal digital assistant, Internet of Things (IoT) device, processing system, wireless access point, router, gateway, and/or any other computing, processing, and/or communication device capable of performing the functions described herein.

The computing device 200 includes a processing device 202 that executes algorithms and/or processes data in accordance with operating logic 208, an input/output device 204 that enables communication between the computing device 200 and one or more external devices 210, and memory 206 which stores, for example, data received from the external device 210 via the input/output device 204.

The input/output device 204 allows the computing device 200 to communicate with the external device 210. For example, the input/output device 204 may include a transceiver, a network adapter, a network card, an interface, one or more communication ports (e.g., a USB port, serial port, parallel port, an analog port, a digital port, VGA, DVI, HDMI, FireWire, CAT 5, or any other type of communication port or interface), and/or other communication circuitry. Communication circuitry may be configured to use any one or more communication technologies (e.g., wireless or wired communications) and associated protocols (e.g., Ethernet, Bluetooth®, Wi-Fi®, WiMAX, etc.) to effect such communication depending on the particular computing device 200. The input/output device 204 may include hardware, software, and/or firmware suitable for performing the techniques described herein.

The external device 210 may be any type of device that allows data to be inputted or outputted from the computing device 200. For example, in various embodiments, the external device 210 may be embodied as one or more of the devices/systems described herein, and/or a portion thereof. Further, in some embodiments, the external device 210 may be embodied as another computing device, microphone, printer, display, alarm, peripheral device (e.g., keyboard, mouse, touch screen display, etc.), and/or any other computing, processing, and/or communication device capable of performing the functions described herein. Furthermore, in some embodiments, it should be appreciated that the external device 210 may be integrated into the computing device 200.

The processing device 202 may be embodied as any type of processor(s) capable of performing the functions described herein. In particular, the processing device 202 may be embodied as one or more single or multi-core processors, microcontrollers, or other processor or processing/controlling circuits. For example, in some embodiments, the processing device 202 may include or be embodied as an arithmetic logic unit (ALU), central processing unit (CPU), digital signal processor (DSP), graphics processing unit (GPU), field-programmable gate array (FPGA), application-specific integrated circuit (ASIC), quantum computing processors, and/or another suitable processor(s). The processing device 202 may be a programmable type, a dedicated hardwired state machine, or a combination thereof. Processing devices 202 with multiple processing units may utilize distributed, pipelined, and/or parallel processing in various embodiments. Further, the processing device 202 may be dedicated to performance of just the operations described herein, or may be utilized in one or more additional applications. In the illustrative embodiment, the processing device 202 is of a programmable variety that executes algorithms and/or processes data in accordance with operating logic 208 as defined by programming instructions (such as software or firmware) stored in memory 206. Additionally or alternatively, the operating logic 208 for the processing device 202 may be at least partially defined by hardwired logic or other hardware. Further, the processing device 202 may include one or more components of any type suitable to process the signals received from input/output device 204 or from other components or devices and to provide desired output signals. Such components may include digital circuitry, analog circuitry, or a combination thereof.

The memory 206 may be of one or more types of non-transitory computer-readable media, such as a solid-state memory, electromagnetic memory, optical memory, or a combination thereof. Furthermore, the memory 206 may be volatile and/or nonvolatile and, in some embodiments, some or all of the memory 206 may be of a portable variety, such as a disk, tape, memory stick, cartridge, and/or other suitable portable memory. In operation, the memory 206 may store various data and software used during operation of the computing device 200 such as operating systems, applications, programs, libraries, and drivers. It should be appreciated that the memory 206 may store data that is manipulated by the operating logic 208 of processing device 202, such as, for example, data representative of signals received from and/or sent to the input/output device 204 in addition to or in lieu of storing programming instructions defining operating logic 208. As shown in FIG. 2, the memory 206 may be included with the processing device 202 and/or coupled to the processing device 202 depending on the particular embodiment. For example, in some embodiments, the processing device 202, the memory 206, and/or other components of the computing device 200 may form a portion of a system-on-a-chip (SoC) and be incorporated on a single integrated circuit chip.

In some embodiments, various components of the computing device 200 (e.g., the processing device 202 and the memory 206) may be communicatively coupled via an input/output subsystem, which may be embodied as circuitry and/or components to facilitate input/output operations with the processing device 202, the memory 206, and other components of the computing device 200. For example, the input/output subsystem may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations.

The computing device 200 may include other or additional components, such as those commonly found in a typical computing device (e.g., various input/output devices and/or other components), in other embodiments. It should be further appreciated that one or more of the components of the computing device 200 described herein may be distributed across multiple computing devices. In other words, the techniques described herein may be employed by a computing system that includes one or more computing devices. Additionally, although only a single processing device 202, I/O device 204, and memory 206 are illustratively shown in FIG. 2, it should be appreciated that a particular computing device 200 may include multiple processing devices 202, I/O devices 204, and/or memories 206 in other embodiments. Further, in some embodiments, more than one external device 210 may be in communication with the computing device 200.

The computing device 200 may be one of a plurality of devices connected by a network or connected to other systems/resources via a network (e.g., devices of the cloud-based system 110 or, more generally, the system 100). The network may be embodied as any one or more types of communication networks that are capable of facilitating communication between the various devices communicatively connected via the network. As such, the network may include one or more networks, routers, switches, access points, hubs, computers, client devices, endpoints, nodes, and/or other intervening network devices. For example, the network may be embodied as or otherwise include one or more cellular networks, telephone networks, local or wide area networks, publicly available global networks (e.g., the Internet), ad hoc networks, short-range communication links, or a combination thereof. In some embodiments, the network may include a circuit-switched voice or data network, a packet-switched voice or data network, and/or any other network able to carry voice and/or data. In particular, in some embodiments, the network may include Internet Protocol (IP)-based and/or asynchronous transfer mode (ATM)-based networks. In some embodiments, the network may handle voice traffic (e.g., via a Voice over IP (VOIP) network), web traffic, and/or other network traffic depending on the particular embodiment and/or devices of the system in communication with one another. In various embodiments, the network may include analog or digital wired and wireless networks (e.g., IEEE 802.11 networks, Public Switched Telephone Network (PSTN), Integrated Services Digital Network (ISDN), and Digital Subscriber Line (xDSL)), Third Generation (3G) mobile telecommunications networks, Fourth Generation (4G) mobile telecommunications networks, Fifth Generation (5G) mobile telecommunications networks, a wired Ethernet network, a private network (e.g., such as an intranet), radio, television, cable, satellite, and/or any other delivery or tunneling mechanism for carrying data, or any appropriate combination of such networks. It should be appreciated that the various devices/systems may communicate with one another via different networks depending on the source and/or destination devices/systems.

It should be appreciated that the computing device 200 may communicate with other computing devices 200 via any type of gateway or tunneling protocol such as secure socket layer or transport layer security. The network interface may include a built-in network adapter, such as a network interface card, suitable for interfacing the computing device to any type of network capable of performing the operations described herein. Further, the network environment may be a virtual network environment where the various network components are virtualized. For example, the various machines may be virtual machines implemented as a software-based computer running on a physical machine. The virtual machines may share the same operating system, or, in other embodiments, different operating system may be run on each virtual machine instance. For example, a “hypervisor” type of virtualizing is used where multiple virtual machines run on the same host physical machine, each acting as if it has its own dedicated box. Other types of virtualization may be employed in other embodiments, such as, for example, the network (e.g., via software defined networking) or functions (e.g., via network functions virtualization).

Accordingly, one or more of the computing devices 200 described herein may be embodied as, or form a portion of, one or more cloud-based systems (e.g., the cloud-based system 110). In cloud-based embodiments, the cloud-based system may be embodied as a server-ambiguous computing solution, for example, that executes a plurality of instructions on-demand, contains logic to execute instructions only when prompted by a particular activity/trigger, and does not consume computing resources when not in use. That is, system may be embodied as a virtual computing environment residing “on” a computing system (e.g., a distributed network of devices) in which various virtual functions (e.g., Lambda functions, Azure functions, Google cloud functions, and/or other suitable virtual functions) may be executed corresponding with the functions of the system described herein. For example, when an event occurs (e.g., data is transferred to the system for handling), the virtual computing environment may be communicated with (e.g., via a request to an API of the virtual computing environment), whereby the API may route the request to the correct virtual function (e.g., a particular server-ambiguous computing resource) based on a set of rules. As such, when a request for the transmission of data is made by a user (e.g., via an appropriate user interface to the system), the appropriate virtual function(s) may be executed to perform the actions before eliminating the instance of the virtual function(s).

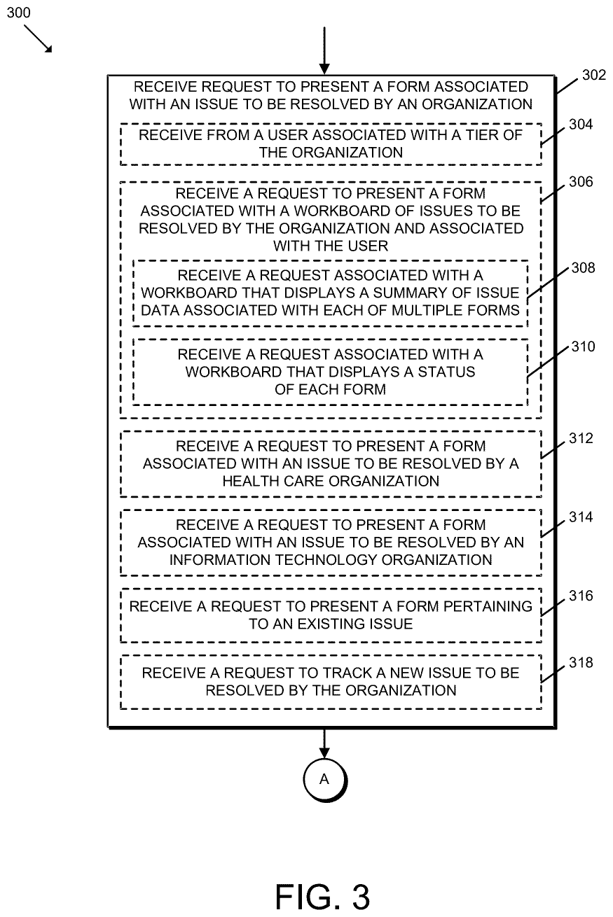

Referring now to FIG. 3, in use, a computing system (e.g., the system 100, including the cloud-based system 110, and/or other computing devices described herein) may execute a method 300 for enhanced issue tracking and resolution. In the illustrative embodiment, it should be appreciated that the method 300 may be executed, in full or in part, by the cloud-based system 110 of the system 100. It should be appreciated that the particular blocks of the method 300 are illustrated by way of example, and such blocks may be combined or divided, added or removed, and/or reordered in whole or in part depending on the particular embodiment, unless stated to the contrary.

The illustrative method 300 begins with block 302 in which the computing system receives a request to present a form associated with an issue to be resolved by an organization. In the illustrative embodiment, and as indicated in block 304, the computing system receives the request from a user associated with a tier of an organization. The user may be a person utilizing a user device 140, 142 and the user device 140, 142 may transmit, in response to a selection of a user interface element presented by the user device 140, 142 to the user, a request through the network 150 to the cloud-based system 110 to present the form. As indicated in block 306, the computing system may receive a request to present a form associated with a workboard of issues to be resolved by the organization and associated with the user. In some embodiments, the computing system may receive a request that is associated with a workboard that displays a summary of issue data associated with each of multiple forms, as indicated in block 308. That is, the workboard from which the request is generated may display, for example, a list of different issues with a summary of a least a portion of the underlying issue data 136 associated with each form. As indicated in block 310, the computing system may receive a request that is associated with a workboard that displays a status of each form, such as when an issue associated with each form was first submitted to the computing system and stored in the issue data 136, a due date associated with each form, and actions taken relative to the corresponding issue by another tier of the organization (e.g., via a corresponding version of the form for that tier).

The computing system may receive a request to present a form associated with an issue to be resolved by a health care organization, such as a hospital, as indicated in block 312. As such, inefficiencies in tracking and resolving the issue may affect the speed or quality of medical services provided to patients. In other embodiments, the computing system may receive a request to present a form associated with an issue to be resolved by an information technology organization, as indicated in block 314. Accordingly, delays or failures in tracking and resolving the issue may negatively impact the operations of a communication network, hardware, and/or software that operates on data communicated via the network. In yet other embodiments, the computing system may receive a request to present a form associated with another type of organization that provides corresponding services or products, such as an organization that manufactures physical products such as equipment, vehicles, appliances, buildings, components of buildings, or other physical products. As will be appreciated, delays or failures in tracking and resolving issues associated with any such organization may have adverse impacts on the operations of the organization and the products and/or services provided by the organization.

As indicated in block 316, the computing system may receive a request to present a form that pertains to an existing issue. That is, the computing system may receive a request to present a form for an issue that has already been identified by a user who entered corresponding issue data 136 for the issue into a corresponding form. That user who initially submitted the issue may be the present user or another user, such as a user associated with a different tier of the organization. Alternatively, and as indicated in block 318, the computing system may receive a request to track a new issue to be resolved by the organization. That is, in such instances, the issue was not previously submitted by another user associated with a tier of the organization.

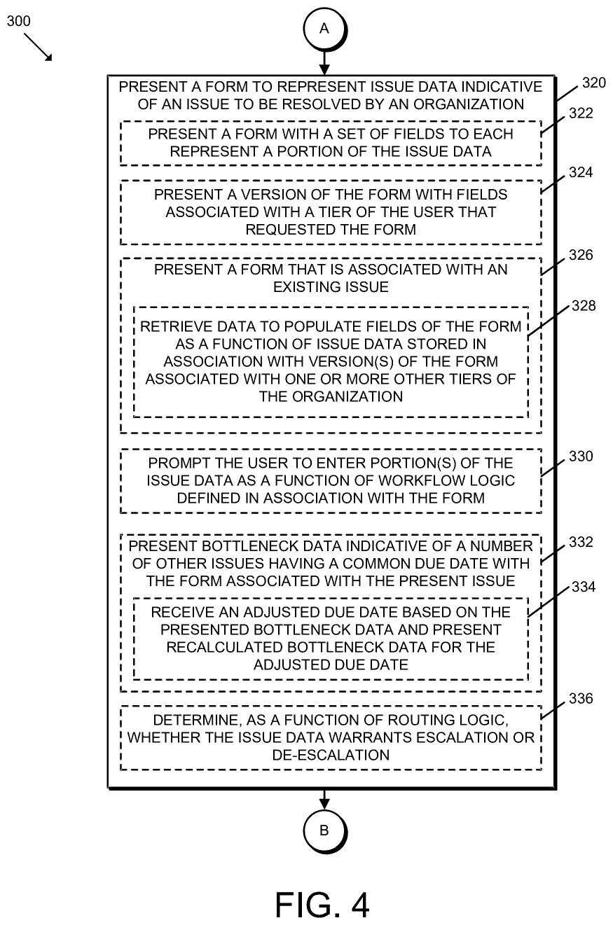

Continuing the method 300, in block 320 of FIG. 4, the computing system presents a form, such as in a user interface, to represent issue data indicative of an issue to be resolved by an organization. That is, the computing system presents the form in response to the request from block 302 of FIG. 3. In doing so, in block 322, the computing system presents a form with a set of fields that each represent or correspond with a portion of the issue data 136 associated with the issue. As indicated in block 324, the computing system presents a version of the form with fields associated with a tier of the user that requested the form. That is, the computing system presents a version of the form that has fields that are determined to be appropriate for the tier of the organization that the user who requested the form belongs to. As described above, in the illustrative embodiment, the computing system may produce different versions of a form, with each version being associated with and having one or more fields that are unique to that version of the form for the corresponding tier. The computing system may present a form that is associated with an existing issue, such as an issue that was previously submitted by a user, as indicated in block 326. In such embodiments, in block 328, the computing system may retrieve data to populate fields of the form as a function of (e.g., based on) issue data 136 that was stored in association with one or more versions of the form associated with one or more other tiers of the organization. For example, if the present user belongs to tier 3 of the organization, the computing system may populate one or more fields of the tier 3 version of the form with issue data 136 that was entered into tier 1 and/or tier 2 versions of the form in association with the issue.

The computing system, in block 330, may prompt the user to enter portions of the issue data 136 as a function of (e.g., based on) workflow logic defined in association with the form. The workflow logic may be stored, for example, in the form data 130, and may define a series of questions, with corresponding user interface elements, such as text fields, drop down menus, radio buttons, or the like. In executing the workflow logic, the computing system may selectively hide or display different sets of such user interface elements as the user progressively enters information (e.g., issue data) into the form, based on the content of the information being provided by the user via the form.

In some embodiments, in block 332, the computing system may present bottleneck data, which may be embodied as data that is indicative of a number of other issues or forms associated with those issues having a common due date with the form associated with the present issue. The due date may be the date by which the underlying issue is to be resolved or a date by which the user must complete all of the applicable fields of the form. As will be appreciated, once the form is completed, the issue may be escalated or de-escalated (e.g., resolving at the present tier and sent down) to another tier, such as based on the routing data 132 described above. In some embodiments, in block 334, the computing system may receive an adjusted due date based on the presented bottleneck data. For example, if more than a defined number of forms are all due on the same date as the present form, the user may select a different due date for completion of the form, as completing the form in addition to the other forms due on the same date may not be feasible. In other embodiments, the computing system may determine an adjusted due date for the present form based on the bottleneck data, such as by selecting a different due date if the number of forms due on an initially determined due date exceeds a threshold number. Regardless, in response to an adjustment to the due date, the computing system may recalculate the bottleneck data for the adjusted due date. In some embodiments, the computing system may repeatedly adjust the due date until the bottleneck data satisfies a threshold number (e.g., is less than the threshold number). In some embodiments, the computing system may present the user with a pop-up window or similar dedicated user interface element allowing the user to view a bottleneck detected by the computing system and/or to adjust dates for one or more forms. In block 336, the computing system may determine, as a function of routing logic, which may be defined in the routing data 132, whether the issue data associated with the present issue warrants escalation or de-escalation. As described above, in at least some embodiments, the issue routing system 122 may determine whether escalation or de-escalation is warranted as a function of the issue data and the routing logic, which may be defined in the routing data 132. In some embodiments, the issue routing system 122 may utilize machine learning models, user input, guided user interface prompts, and/or other methods in making this determination and/or performing other functions. In some embodiments, the issue routing system 122 may automatically adjust dates, escalate, and/or de-escalate issues based on bottleneck data. In other embodiments, the issue routing system 122 may present the user with a prompt or notification allowing the user to take action in response to a detected bottleneck.

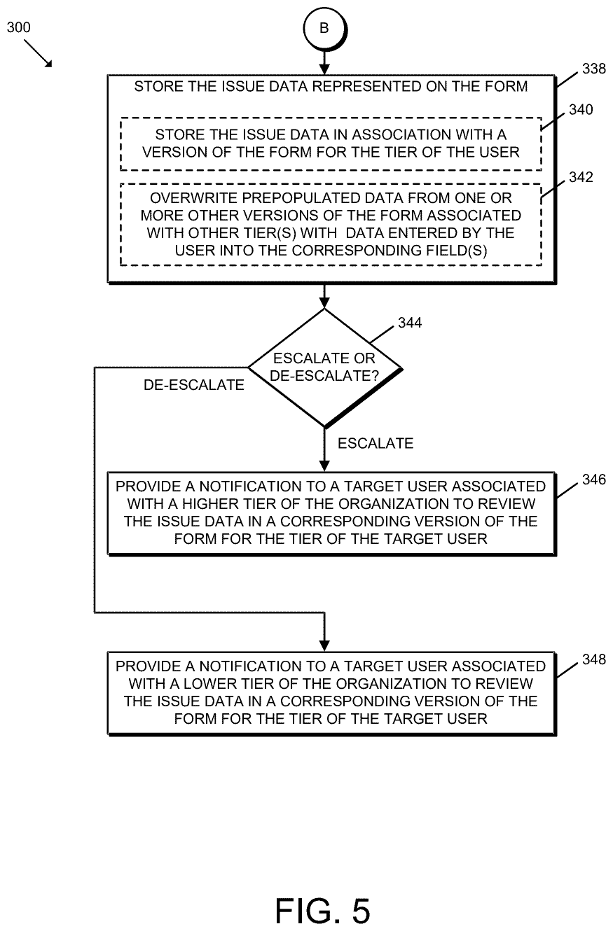

Continuing the method 300, in block 338 of FIG. 5, the computing system may store the issue data represented on the form, such as in the issue data 136. In doing so, in block 340, the computing system may store the issue data in association with a version of the form for the tier of the user. Further, the computing system may replicate, copy, or otherwise enable a least a portion of that data to be incorporated into a version of the form associated with another tier of the organization. As described above, when presenting a form associated with a given tier, the computing system may prepopulate one or more fields represented on the form with issue data associated with other versions of the form associated with other tiers of the organization. In block 342, the computing system may overwrite any prepopulated data from one or more other versions of the form associated with one or more other tiers with data entered by the user into the corresponding fields of the version form associated with the tier of the user. That is, if a field was prepopulated with data from another version of the form associated with another tier of the organization (e.g., in block 328) and the present user changed that data, then in the version of the issue data stored by the computing system in association with the present version of the form (the version of the form associated with the tier of the present user), the computing system stores the data that was provided by the present user, rather than any data that originated from another version of the form associated with another tier. However, if the version of the form for that other tier is requested (e.g., by a user associated with that tier), then the computing system presents the issue data that was stored in association with the fields for that version of the form, even if that data is different from the same fields in the version of the form for another tier. As an example, if a tier 1 user enters value A into field A into the tier 1 version of the form, then a tier 2 user views a tier 2 version of the form, which pulls in value A into field A of the tier 2 version of the form, and the tier 2 user changes value A to value B in field A of the tier 2 version of the form, then value for field A will be different for the tier 1 version of the form and the tier 2 version of the form, as the computing system stores the fields separately, on a per form and per tier (e.g., version) basis. In some embodiments, the ability of a user to overwrite a prepopulated field in a version of a form may be restricted by, e.g., tier of the user, comparison of the tiers of two or more versions of the form, user role, user location, specific user permissions, specific form configurations, etc. In at least some embodiments, one version of a form may selectively inherit data or other contextual information from another version of a form.

Certain embodiments may allow specially authorized users to override fields in forms created by other users. Such override permissions may be defined by one or more of, e.g., the user's role, tier, department, location, special permissions, the field being overridden, etc. In some embodiments, overriding a field in a given form will automatically override that field in any related forms and/or system views.

In block 344, the computing system determines the subsequent course of action based on whether the computing system determined to escalate or de-escalate the issue. De-escalation may represent a determination that the issue has been resolved. In response to a determination to escalate the issue, the method 300 advances to block 346, in which the computing system may provide a notification a target user associated with a higher tier of the organization to review the issue data in a corresponding version of the form for the tier of the target user. For example, the computing system may provide the notification as an entry in a workboard displayed to that target user. Conversely, if the computing system determines to de-escalate the issue, the method 300 advances to block 348, in which the computing system may provide a notification to a target user associated with a lower tier of the organization to review the issue data in a corresponding version of the form for the tier of the target user. For example, the computing system may provide the notification by displaying an entry associated with the issue in a workboard displayed to the target user. In either case, the computing system may implement downward routing including contextual guidance and/or instructions when an issue is de-escalated. The system may transmit, e.g., ownership, directions, required actions, expectations, and/or instructions which may help the receiving user and/or tier address the issue effectively and prevent unnecessary re-escalation.

In certain embodiments, one or more external systems may generate issue data for representation as a form. For example, a platform may be configured to interface with other existing systems across different domains and receive externally generated risk or issue signals. The platform, in some embodiments, may not calculate or diagnose risk itself, but instead may ingest structured risk indicators, scores, or flags produced by those systems and convert them into actionable items. When a risk is received, the platform may automatically create an entry on one or more associated workboards, including relevant context about what is driving the risk, so it can be reviewed, managed, and escalated if necessary. For example, in healthcare, an electronic medical record may identify a patient as high risk based on documented criteria, and that risk information can be interfaced into the platform and auto-populated onto a Tier 1 workboard for operational review. Some embodiments may apply this same integration pattern to other industries, allowing externally identified risks to be visible, trackable, and manageable within the tiered workflow without manual data entry. The platform may also integrate with other systems to bring in any organization-defined data, events, or signals the organization wants surfaced for review, such as safety events, performance counts, operational issues, or other tracked topics. Externally sourced items may then appear in the platform to be visible, reviewed, discussed, escalated, and managed through a tiered huddle workflow without manual data entry, based on how the platform is configured.

It should be appreciated that in some embodiments the system may be configured to allow users to create forms at one or more tiers. The tiers a given user may create a form for may be defined by, e.g., the user's role, department, location, special permissions, etc. For example, the system may be configured so an IT senior director may submit a ticket directly to the president's tier to address a security breach. Similarly, in embodiments where one or more external systems may generate issue data, each of the one or more external systems may be configured to generate tickets of one or more respective tiers.

It should be appreciated that in some embodiments the organization may be a healthcare organization in which medical services are provided to patients by personnel of the organization. The personnel may include frontline caregivers and issues may be identified and addressed in connection with unit or department level huddles, in which personnel of the healthcare organization share status updates and information regarding medical services to be provided for patients. Such huddles may be used to identify potential risks, concerns, or operational system failures that adversely affect patient outcomes and that may require swift escalation to an appropriate leadership tier for timely coordination and resolution. As such, when encountering issues that adversely affect the healthcare organization to deliver medical services, the method's enhanced escalation, tracking, and resolution of such issues thereby enabling more efficient delivery of care and improve patient outcomes.

Embodiments of the technologies are disclosed in more detail in the following description. In the following description, embodiments of the system, the user interfaces presented by the system, the methods and operations executed in connection with the system may be referred to as the “CIRC Form” or “Collaborative Interconnected Response & Coordination Form.”

The CIRC Form represents a transformative approach to managing complex tasks and resolving issues across multi-tiered teams, departments, or users. It replaces the traditional notion of collaboration, where multiple users contribute to one shared form or static document, with a dynamic, interconnected network of individualized forms. Each assigned action owner or team is given their own unique CIRC Form, which is linked directly to the forms of all other entities involved in the issue. These forms are not isolated records; instead, they are active, bi-directional, intelligent points in a collaborative network. Together, they form a fluid and fully traceable chain of communication, updates, interventions, and resolutions.

The architecture stands in sharp contrast to conventional project management tools such as Google Docs, Excel spreadsheets, generic task platforms, and the tier-based forms commonly used in IT Service Management (ITSM) or High Reliability Organization (HRO) Tier Huddles. While such systems provide a surface-level sense of collaboration through shared access and manual editing, they quickly fall apart under the weight of complex, multi-tiered workflows. ITSM platforms, for example, route Incident, Request, Problem, and Change forms through siloed Tier 1, Tier 2, and Tier 3 structures. Updates are typically segmented by role, and visibility is gated by user assignment. Though effective for maintaining audit trails, the structure lacks the transparency, adaptability, and real-time coordination required for true cross-functional engagement. In contrast, CIRC-powered forms are intentionally built for synchronized collaboration across all levels and teams. Each form dynamically connects to others involved in the process, ensuring that every participant, from intake to resolution stays informed and aligned in real time.

In high-stakes environments like healthcare systems, enterprise IT operations, and complex service delivery models, reliance on static, centralized documentation becomes a critical vulnerability. Legacy tools offer no embedded workflow logic, no mechanism for real-time, cross-role coordination, and no intelligent guidance tailored to each contributor's responsibilities. Instead, they demand constant manual oversight, fragmented communications, and error-prone interpretation of outdated or siloed information. Visibility is constrained by rigid permissions, and accountability is often lost in disconnected update chains. CIRC replaces this outdated paradigm with a living, interconnected framework.

CIRC Forms are architected as living, role-aware systems that intelligently adapt to the user's place in the workflow. Each CIRC Form acts as both a command center and a communication node, automatically surfacing relevant actions, cascading updates across linked forms, and ensuring that no information gets siloed. By unifying visibility, action, and accountability in a single interconnected structure, CIRC eliminates the friction caused by traditional ticketing hierarchies and static documentation. CIRC redefines coordination as a real-time, horizontally synchronized process where information does not just flow, it evolves transparently alongside the work itself. This paradigm shift transforms how organizations manage complexity, empowering teams to respond with precision, agility, and shared understanding from intake through final resolution.

CIRC enables dynamic logic that powers real-time workflow navigation. A feature in CIRC is the configurable algorithm decision guided elements built into each form to drive the workflow. These logic-based agents respond to users' input in real time, guiding what should happen next based on rules and context. Instead of following a fixed path, the form adapts as information is added, ensuring correct steps, alerts and visibility are triggered automatically. This helps guide the user through the decision-making process, helping to standardize workflows, reduce drift and improve accuracy and performance.

Further, CIRC is organized by logic and optimized for focus. In conventional platforms, what begins as an “everyone-in-one-doc” or “everyone-in-their-own-doc” setup quickly turns into a chaotic maze of overlapping edits, scattered comments, and disconnected threads. In shared documents, too many hands in a single file can create version confusion like lost context, and conflicting inputs. Conversely, when each team or user works in their own isolated document, collaboration fragments even further. Teams often complain of notification overload, unclear handoffs, and a lack of visibility into upstream or downstream actions. Instead of supporting complex workflows, these tools rely on humans to bridge the gaps, leading to misalignment, delays, and rework. CIRC, on the other hand, combines the best parts of both and removes the negatives of each.

CIRC Workboards solve this by embedding logic-driven organization directly into the system architecture. Tasks, updates, and priorities are automatically surfaced to the top of the Workboard and to the right people at the right time, without the user having to dig. Instead of static lists or buried comment chains, CIRC's dynamic Workboards are automatically organized based on configurable inputs, ensuring that what's relevant is always front and center. With CIRC, work comes to the user, not the other way around, and is delivered with clear visibility into all related updates, decisions, and contributors. Escalation pathways are built-in, role-aware, and fluid, keeping everyone aligned from first identification to final resolution.

CIRC utilizes a precision architecture for tiered systems. In tiered structures, conventional platforms and static forms become especially hazardous. Tier 1 may identify the issue, Tier 2 intervenes, Tier 3 resolves, and executive leadership may later need to assess broader patterns or systemic risk. Yet a static document provides no reliable way to move that information smoothly across layers. Communication becomes dependent on manual updates or side conversations, and downstream teams often lack visibility in upstream decisions. Just as critically, upstream teams have no way of knowing whether downstream actions have fully resolved the issue or if anything was missed.

Systems like Google Docs or shared workspaces and ITSM platforms treat access as all or nothing. Everyone sees the same file, even if they should not, or access is restricted so tightly that workflows break down. This creates a frustrating choice: either overexpose sensitive information to people who do not need it or silo it so completely that key team members cannot contribute. The result is confusion, delays, and unclear ownership, especially risky in fast-moving environments like hospitals or enterprise support teams.

The CIRC Form solves this problem with flexible, role-based visibility and editing control. Access is configured based on the user's function. For example, a frontline nurse does not need the full workflow audit trail; she needs to know her alert was seen, understood, and acted on. A Tier 3 IT lead does not need a checklist; he needs real-time visibility into every action taken, what has worked, what has failed, and where the issue still lives, so he can pick up the baton and drive resolution without wasting time retracing steps. Treating all users as if they operate in the same context dilutes precision, slows action, and breaks the chain of accountability.

CIRC can safeguard protected information while still allowing access to collaboration. A Tier 1 nurse manager can view pertinent information on higher-tier forms but cannot fully access and make edits to them. Meanwhile, Tier 4 users can both fully view and edit Tier 1 forms, allowing leadership to intervene, update, or override as needed. Fields within the forms can also be selectively hidden or shown depending on job type or clearance level. A documentation scribe, for instance, may have broader editing rights than a unit manager because their role involves validating records across multiple tiers. This approach supports what static systems cannot: safe, transparent collaboration with just the right level of access. It gives each user a clear window into what's happening above and below their tier, without compromising data integrity or security. With CIRC, workflows stay intact, context is preserved, and accountability is assigned at every level.

CIRC transforms the system. This is where CIRC, embedded within platforms like InSight, delivers a radically different approach. Rather than expecting users to chase information across disconnected tools, CIRC brings the correct information to the right user, automatically, contextually, and with precision. Every tier receives a tailored form, role-specific, logic-driven, and interconnected. These forms evolve based on input, escalate by rule, and share critical progress bi-directionally with all stakeholders. Nothing is lost in handoff, and no team is left blind to the status of their dependencies.

Because each user's form contributes structured data into a central logic model, updates are instantly reflected across interconnected forms and personal Workboards. Unnecessary fields are hidden to deliver concise focused information. This means users see a clear, curated view of what's assigned to them, without the noise of irrelevant information. Sensitive data is intelligently hidden or filtered to meet compliance needs without breaking visibility. Status changes become triggers, not footnotes. Delays are surfaced system-wide without anyone needing to forward a spreadsheet or manually distribute updates.

CIRC provides a predictive intelligence through click-driven framework. A feature that sets CIRC apart is not only its ability to organize real-time collaboration, but its capacity to generate high-integrity data through the very clicks that guide users through each form. Every escalation, resolution status, category selection, or handoff is captured as a structured data point that is uniform, trackable, and ready for synthesis. This click-based architecture is discrete, quantitative data, rather than free text, qualitative data. CIRC enables users to analyze both, but quantitative is much easier and faster to work with and provides structure to make sure all users are answering the same questions, with the same terms, and same guidance. Not only is CIRC user-friendly and quick but it also eliminates the ambiguity of free text and transforms frontline activity into clean datasets that fuel real-time Workboards and enterprise-level insight.

From these inputs, leadership can surface emerging risk themes, spot unresolved clusters, track performance trends across teams, and even build predictive models to anticipate where breakdowns are likely to occur. This capability is not retrospective; it's active. Delays are flagged before they compound. Underperforming workflows become visible before they result in harm. CIRC does not just reflect what has happened; it reveals what's about to happen, empowering organizations to allocate resources, standardize interventions, and make decisions with foresight rather than hindsight. In this way, CIRC bridges the gap between real-time operations and long-range strategy, all through the silent intelligence of structured user interaction.

Unlike a Google Doc, which holds information but does nothing with it, CIRC activates information. It transforms forms from static pages into dynamic engines of clarity, momentum, and collaboration. It preserves the whole narrative, what was done, who did it, and what happened next, ensuring that complex handoffs don't become dead ends.

This innovation is proven in practice, not just theory. The CIRC platform has been successfully implemented in 4 hospitals and over 300 ambulatory practices, where it has processed and resolved close to 10,000 escalations through a five-tier safety huddle system and is currently processing nearly 3,000 submissions and escalations per month. Escalations flow both upward and downward across all five tiers, enabling real-time resolution and visibility throughout the structure. End users report that the platform is intuitive, fast, and dramatically more straightforward to use, thanks to its click-based, low-friction interface. Manual entry is minimized, escalation logic is built in, and users can view the entire history of any issue across all tiers.

As a result, frontline and leadership teams remain aligned without needing duplicate systems or status check-ins. Uniquely, Tier One users can view activities, actions, and outcomes occurring at higher tiers, a capability made possible by interconnect CIRC forms. This ensures that frontline staff remain connected to the whole resolution process, not just their initial escalation. Based on this success, CIRC is now being expanded across all units within 9 Commissioner hospitals, with further rollout underway to ancillary and shared services. Data collected through the platform is continuously analyzed to generate actionable, system-wide insights. This represents one of the most significant safety huddle transformations in the nation, underscoring the platform's ability to scale across complex, multi-site healthcare systems.

CIRC does not just solve the problem of convenience; it solves for clarity, accountability, and forward motion. It replaces guesswork with guidance, fragmentation with fluidity, and passive documentation with active orchestration. In doing so, it redefines what digital collaboration should look like in complex high-reliability systems where precision isn't optional, it's mission-critical.

The following is a description of scenarios in action. The following scenarios showcase how the CIRC innovation streamlines HRO tiered escalation and resolution of safety incidents within a multihospital health care organization. The CIRC innovation is proven to be successful through a test of concept with over 16,000 escalations in one of the largest healthcare organizations in the country. CIRC acts as a centralized brain, bringing information directly to each user, maintaining full historical context, and guiding decisions in real time. These examples illustrate how CIRC transforms frontline observations into coordinated, system-wide action.

Application to tiered safety huddles is described herein. With regard to unit/practice level huddle enhancement, the unit/practice level Tiered Safety Huddle utilizes a Digital Agenda module specifically designed to enhance unit and practice-level huddles with frontline associates. The Digital Agenda provides a structured, interactive platform that ensures both upward escalation and downward communication are seamlessly integrated into daily safety practices. The Digital Agenda may be accessed through an icon on a desktop or laptop computer, a mobile application on a cellular device, or a QR code that frontline associates scan to instantly open the agenda on their device. These flexible access options ensure that the tool is available in real time across diverse work environments.

With regard to standardization and routine reliability, the Digital Agenda ensures that unit and practice huddles are completed routinely each day as intended, preventing process drift. By delivering a standardized, step-by-step agenda from the organizational gateway down to every practice and unit, the system guarantees reliability across all tiers. This ensures that daily tiered huddles are consistent, structured, and aligned with organizational expectations.

Importantly, the Digital Agenda makes it simple for any leader or associate to facilitate a huddle. By following the guided prompts within the tool, even new or temporary facilitators can confidently lead discussions. This feature is particularly critical for large-scale organizations—such as those operating over 300 ambulatory practices across a state or region, in addition to multiple hospitals with numerous inpatient units—where consistency and sustainability are often difficult to achieve.

With regard to frontline participation, the Digital Agenda enables direct participation by frontline associates. During huddles, associates may access the Tiered Safety Escalation Form to submit safety events, recognitions or concerns directly into the agenda, escalate issues into the CIRC workflow for immediate manager review, and view previously resolved or open escalations relevant to their practice. This bidirectional functionality ensures frontline insights are captured at the source and immediately routed into the broader escalation process.

CIRC provides a communication hub. The Digital Agenda also serves as a centralized communication tool. Huddle leaders can present and share organizational updates directly through the agenda, including: links to policies, procedures, and resource documents, embedded images, diagrams, and media to support clarity, scripted messages or updates for leaders to read aloud, ensuring consistency across all frontline teams, and direct communications from upper-tier leadership, including safety alerts, lessons learned from events and time-sensitive updates that are delivered directly through the agenda to frontline associates. This ensures that critical messages are disseminated reliably and promptly across the organization, bridging the gap between executive leadership and frontline staff.

With regard to safety metrics integration, the agenda may also display real-time safety metrics, such as counters showing the number of days since the last event, ongoing compliance rates, or trend indicators. These metrics are configurable to organizational preferences and reinforce situational awareness at the unit level.

CIRC also provides cultural reinforcement. The Digital Agenda integrates HRO principles into its structured framework, facilitating both retrospective review and forward-looking planning. By embedding cultural expectations directly into the daily workflow, the agenda ensures sustained alignment with organizational safety values and supports the long-term resilience of high-reliability practices.

CIRC also provides compliance and verification. In at least some embodiments, all interactions with the Digital Agenda are logged, including associate submissions, huddle completions, and acknowledgement of communications. This allows the organization to confirm and track that huddles are being conducted daily and consistently, frontline associates are actively participating, and communication is being cascaded effectively to the point of care.

CIRC facilitates frontline identification and intake with a common starting point. With regard to issue reporting, a frontline associate identifies an issue during the unit/practice huddle or at anytime during their shift and submits it using the Tier Safety Escalation Intake Form, accessible via a desktop icon on every hospital computer or within the Digital Huddle Agenda. The intake form is user-friendly, using a click-through process that enables frontline associates to submit escalations in under a minute, supporting sustainment. The associate selects their manager from a dropdown menu, which uses automated routing logic to direct the issue to the correct leader. Alternatively, the automated routing logic may direct the issue based on a user's stored manager, location, or other data. With regard to manager access, the manager receives an alert, clicks their desktop icon, and is logged directly into their personal InSight landing page. From there, they can access resources and their personalized Workboards, which are powered by smart logic to keep issues automatically organized. As part of the daily tier safety huddle workflow, managers also review their Active Workboard each morning for new issues.

CIRC Workboards serve as the command center for each user, keeping escalations visible, organized, and prioritized. The Workboards are dynamic, logic-driven, and update automatically as issues move through the workflow. Each user's view is personalized to their role, showing only what requires their attention while maintaining visibility across linked tiers. With regard to workboard views, each tier has a set of specific Workboards intentionally created with logic to keep issues organized automatically. An active workboard displays newly escalated issues and items currently being addressed. Each row links directly to the CIRC form for quick action. Updates entered by connected users appear instantly and are highlighted until acknowledged, ensuring no progress or communication is overlooked. A queue (e.g., a queue workboard) lists items with a set follow-up date. On that date, they automatically reappear on the Active Workboard for review, with reminder alerts sent in advance. A Continue to Track Workboard provides visibility into issues escalated to higher tiers or other departments. This view is strictly for monitoring progress. That is, users cannot take direct action here, but they can see and/or provide real-time updates to and from the current issue owner which may be highlighted. For example, a manager could provide an update to a director regarding low stock of an item if shipment arrives/stock runs out completely. All users who have interacted with an issue can add new data to their form at any time and all other involved users are notified/have that new data highlighted for visibility A Resolved Workboard stores resolved or archived issues. Resolved items remain in the system and can be reactivated at any time, retaining their full historical record for continuity.

In a pathway referred to herein as “Pathway One: Tier 1 Contained Resolution”, CIRC determines that an issue can be resolved at Tier 1. In a Tier 1 Review and Initial Processing phase, each row on the Workboard represents an issue, with the Tier 1 CIRC form linked in the first column. The CIRC form auto-populates the Workboard with key details, such as unit, escalation type, reporter, and description, so the manager can at a glance review the escalation from the Workboard. That is, no manual entry is needed. The managers click directly into the form linked in the first column and begins to review and act. The manager is working within the Tier 1 CIRC form where there are data point fields to click through (category, priority, status) and guided algorithms and coaching prompts to help determine whether the issue can be resolved at Tier 1 or needs escalation.

In this case, the algorithm confirms that the broken ice dispenser can be resolved at Tier 1, avoiding unnecessary escalation. The manager assigns themselves as Action Owner, selects the equipment management team as Point of Contact (POC) from a dropdown (with type-ahead directories), sets a follow-up date, and adds notes. Guided prompts assist the manager in selecting the appropriate POC to engage.

With regard to automated assignment and collaboration, upon saving, the system generates a linked CIRC form for the POC and automatically places it on their Workboard and notifies the POC, while the manager's CIRC form remains visible on their Active Workboard. Relevant details (category, description, unit, priority, manager's name) carry over automatically, saving time with reentries.

The manager's and service team's forms remain interconnected, with updates flowing in real time. By clicking into their own CIRC form, each can immediately see the other's entries. When the service team member or manager records an update, it is highlighted on the other's Workboard until reviewed and acknowledged. This structure ensures bi-directional visibility without duplication of effort.

With regard to notifications and safeguards, email alerts remind Action Owners and POCs ahead of follow-up dates. Further, updates can be entered directly into the CIRC form, as well as shared during huddles. Irrelevant fields remain hidden, keeping communication concise. If a manager fails to act within the designated timeframe, the system alerts their director.

With regard to service team resolution, the equipment team documents in their CIRC form that they will repair the ice dispenser by the end of the day. This update instantly updates the manager's linked view. Each update remains highlighted on the manager's Workboard until acknowledged, ensuring nothing is missed. The manager reviews the service team's updates, acknowledges the highlights to clear them, and confirms completion. Once the repair is completed, the service team member marks the issue as fixed on their CIRC form and moves it to their resolved workboard to archive, triggering an update alert to the manager.

With regard to manager confirmation and closure, resolution alerts are automatically triggered across all connected forms eliminating the need for the service manager to take additional steps to send out communication to the Manager. The issue is flagged on the manager's Active Workboard, signaling that the issue has been marked as resolved by the service team. The manager then clicks into their CIRC form and reviews the service team's CIRC forms resolution description and changes the issue's status to Resolved with a resolution note (“ice dispenser fixed”). Within the CIRC form, and with only a few clicks, the manager can generate a communication report to relevant teams or leaders, sent via email and/or in-platform notifications. The resolved issue moves into the manager's Resolved Workboard. The platform has a section for recent and past communication, generating reports showing communications within a certain time period based on configurable parameters. Past communications may be archived within a configurable rolling calendar. Recent and past reports show all the same fields described in the original communication report.

In a pathway referred to herein as “Pathway Two: Multi-Tier Escalation”, CIRC determines that the issue must be escalated beyond Tier 1. With regard to manager review and algorithm guidance (Tier 1), the manager opens the Tier1 CIRC form listed on their Active Workboard and reviews the associate's safety alert submission, describing a malfunctioning supply, including the lot numbers. The manager completes required fields (e.g., category=supply, priority=high). These fields are configurable per organization and auto-populate across all connected forms. The manager begins answering the algorithm questions within the Tier 1 CIRC form. The questions may include: “Do you have the needed resources to resolve this at your tier?”, “Is the issue potentially happening in other areas?”, “Is this a system issue you are unable to contain?” Based on responses, the system provides a pop-up recommendation to escalate to the next tier. The manager selects their Tier 2 director's name from a dropdown. The issue is automatically sent to the director's Tier 2 Active Workboard, and the Tier 1 CIRC form for this issue is moved to the manager's Continue to Track Workboard for visibility and ongoing monitoring.

With regard to Director Review (Tier 2), the director opens their Active Workboard and clicks on the linked CIRC form. They see a condensed historical record that indicates: The frontline submission, the manager's entries and notes, and auto-populated fields from the manager's entries (category, priority, unit, escalation type). Algorithm questions guide the director, just as they did for the manager. A pop-up directs the director to escalate further. If needed, the director can update any field with prepopulated information and the latest entry will carry over to all linked forms. The director escalates to Tier 3 (Vice President), selecting the appropriate VP or service line (e.g., Neuro, Heart & Vascular). Meanwhile, both the manager and director can track progress and provide updates from their Continue to Track Workboards.

With regard to Vice President Review (Tier 3), the VP reviews the issue from their Active Workboard and opens the Tier 3 Supply Issue CIRC form. The CIRC form displays: Submissions from Tier 1 and Tier 2 and key historical information, condensed for quick review. The VP sees a highlighted update alert indicating that the Tier 1 manager has submitted new information. The update states that the supply with the specified lot numbers has been removed from the unit's circulation. The huddle scribe marks the update as reviewed, which removes the highlight and auto-populates this acknowledgment across all connected CIRC forms, informing the manager that their update has been seen. Guided by algorithm prompts, the VP is prompted to escalate to the next tier due to the potential impact across multiple hospitals. The VP escalates the issue to Tier 4 (Hospital President) and writes in notes “concerned for potential risk across the system” and moves the Tier 3 CIRC form to their Continue to Track Workboard for ongoing visibility.

With regard to President Review (Tier 4), the hospital president opens their Active Workboard and clicks into the issue's CIRC form. They immediately see a concise history of prior tier's submissions and decisions. All pertinent information (category, priority, subject, ticket number) is auto-populated, ensuring accuracy, eliminating the “game of telephone” effect, and saving time. Nothing has to be re-entered, details remain consistent across all tiers, and time is not wasted tracking down information. The president conducts their review, supported by algorithm questions and decision pathways, then escalates the issue to Tier 5 (Organizational President) and moves the Tier 4 CIRC form to their Continue to Track Workboard for ongoing visibility.

With regard to Organizational President Review (Tier 5), the issue escalates to Tier 5, where the organizational president meets with all hospital presidents. The Active Workboard sorts escalations automatically: Critical-priority issues, such as this supply failure, are flagged and listed at the very top to ensure immediate attention. Lower-priority issues are sorted below. As an example, a hospital president reports that a malfunctioning supply may affect all 15 hospitals. Lot numbers are visible on the CIRC form. Supply leadership in the huddle can immediately confirm the affected lots. The organizational president assigns an Action Owner and Points of Contact (POCs) using dropdown menus. A scribe documents the discussion within the CIRC form, supported by facilitator coaching prompts that standardize huddle practices and prevent drift. Such a scribe may be human or an automated system, such as an artificial intelligence model. A target follow-up date is assigned, ensuring accountability. The CIRC form includes a bottleneck feature, displaying a window next to the target date that shows how many follow-ups are already scheduled for that date, helping balance workload.

Upon saving the Tier 5 CIRC form: Action Owners and POCs receive their own CIRC forms on their Workboards to manage resolution. Their actions and updates are visible in real time to Tier 5 leadership and across all linked tier forms. The issue automatically moves into the Huddle Follow-Up Queue and reappears on the Active Workboard once the target date is reached. This keeps the Active Workboard concise, displaying only the submissions that require attention on that day, reducing time spent searching and minimizing confusion. Action Owner alerts are sent via email and posted in the InSight platform, reminding responsible individuals to be prepared to provide updates at the designated time.

With regard to escalation models, linear and/or concurrent models may be used. Tiers may operate as a linear handoff when appropriate, but they are not limited to that approach. Tiers may also operate concurrently, with each level addressing resolution within its scope while escalating the issue for broader awareness, depending on, e.g., organizational configuration and the nature of the issue. For example, an issue may be submitted and routed to Tier One, where a nurse manager reviews it and completes guided assessment questions within the form. Based on those responses, the system may present recommended actions-such as removing a defective supply, capturing supporting images, and notifying risk management- and prompts consideration of escalation if the issue may impact multiple areas. The nurse manager may assign herself as the action owner, set a target date, confirm the suggested steps, and/or escalate the issue to her director. Before submission, the system may confirm that the issue will remain on the nurse manager's Active Workboard while local remediation continues, even as it is escalated for broader awareness. Local actions proceed to completion and are resolved at the Tier One level. In parallel, the issue advances through higher tiers, where leadership may coordinate broader actions, such as issuing safety alerts across facilities. These higher-tier activities do not overwrite or negate completed local remediation. When higher tiers adjust fields such as priority, category, or target timeframe, the system notifies lower tiers to maintain shared awareness, while preserving lower-tier entries for transparency and auditability. For reporting and analytics, values recorded by the tier responsible for system-level resolution may be treated as authoritative, while historical tier-specific inputs remain available for review. This example workflow demonstrates that escalation and remediation may occur sequentially and/or in parallel, supporting timely action, visibility, and accountability without forcing a single operating model.

Additional Features may include features related to using the Active Workboard to drive huddles. At each tier (Director, VP, President, and beyond), huddles are organized and facilitated through the Active Workboard, which serves as the central display board. During each huddle, the Active Workboard is displayed. Issues are automatically sorted by priority, ensuring the facilitator begins with the most critical items at the top and works down the list. This structured approach keeps discussions focused and ensures urgent risks are addressed first. Leaders review escalated items directly in the system, ensuring decisions are based on real-time, interconnected information. This structured approach provides clarity in that each tier sees only the pertinent details they need, continuity in that all historical actions remain linked and accessible, and accountability in that unresolved issues remain active and cannot be prematurely closed by the tier responsible for resolution.

The applicable huddle participant from the department that submitted the initial escalation provides a verbal summary of the issue description. Huddle participants can review the CIRC form and historical information while listening to the report-out. The facilitator (tier leader) guides the discussion, as follows: Tier 2: Director with managers, Tier 3: VP with directors, Tier 4: Hospital president with VPs, and Tier 5: Organizational president with all hospital presidents. After report-outs, the facilitator answers algorithm-driven questions within the CIRC form. At the same time, a scribe uses a dynamic click-through workflow to document the discussion and determine whether to resolve or escalate further. This methodology, with limited free text, enables the scribe to move quickly through the form while ensuring all information is captured accurately.

The structured data captured through this method feeds into dynamic Workboards, which can be analyzed to identify common themes and to create predictive models that enhance proactive risk management and system process improvements.

CIRC enables immediate mitigation communication. At any tier, if the issue requires immediate action, the facilitator selects Yes in response to the CIRC form prompt: Does this issue require immediate mitigation? This opens a communication text box where the scribe enters the mitigation instructions. The scribe then selects the hospitals to notify by checking the hospital and department names from a dropdown list. Once submitted, the communication is distributed through connected distribution lists to send automated email alerts and in-platform notifications for the selected hospitals (tier 5) or departments (tier 4). Communication is effectively sent back down the tiers. Leaders at all levels (managers, directors, VPs, and presidents) receive a Critical Communication Alert. This targeted, system-wide communication is generated and distributed directly from within the CIRC form with just a few clicks, ensuring a fast, accurate, and seamless operation.

CIRC also enables Bi-Directional Escalation (Send Back Down). If an issue is escalated inappropriately, or if an upper-tier facilitator determines that the issue can be resolved at a lower tier, the workflow allows the issue to be sent back down. The scribe selects the status “Send Back Down” in the CIRC form. A text box automatically opens, prompting the facilitator to provide instructions for the lower tier on how to resolve the issue. The scribe enters the instructions, and once submitted, the upper-tier CIRC form is closed and no longer appears on that tier's Active Workboard. Further, the issue automatically reappears on the lower tier's Active Workboard, highlighted as “Sent Back Down.” Additionally, the instructions provided by the upper tier are auto-populated on the lower tier's CIRC form, ensuring clarity and accountability. The lower tier reviews the instructions and proceeds with resolution. If the lower tier later determines that re-escalation is necessary, they may escalate again, but only after attesting that they have completed the instructions provided by the upper tier. This functionality demonstrates that CIRC forms are fully bi-directional, retaining transparency of information while moving up or down the tier structure as needed, so that issues are always addressed at the most appropriate level.

An example flow relating to containment and next steps is described below. In this example, the organizational president issues an immediate mitigation order: All hospitals must remove the affected lot numbers from circulation immediately. The supply leader commits to running a report to identify affected locations and to contact the manufacturer. Within four hours (7 a.m.-11 a.m.), the escalation has reached the CEO (Tier 5), triggered organization-wide mitigation across several hundred ambulatory practices and 9 hospitals, and activated supply chain leaders to analyze and contain the risk.

With regard to tracking and visibility across tiers, the issue remains visible across all tiers: Managers and directors can monitor Tier 5 actions via their Continue to Track Workboards and provide updates to upper tiers. Each tier can click into their linked CIRC form to see what higher tiers are doing in real time. Likewise, upper tiers can monitor the actions of Action Owners and POCs, seeing updates as they occur. Configurable security controls ensure visibility without overreach. Lower tiers can review but not edit upper-tier forms. Organizations can configure what sensitive information is visible at each level. This ensures bi-directional transparency: upper tiers can see what lower tiers are doing, and lower tiers can see what upper tiers are doing, with visibility through configurable security controls to protect sensitive information. Additionally, CIRC forms may operate as a quality protected platform supporting a focused view. For example, when a user logs in, they may see only their specific assignments or workboards. If cross-coverage is needed (e.g., covering for a manager on vacation), the user can switch out of the focused view and access a broader view to see and manage another manager's workboard, subject to role-based access controls.

Regarding closure and reactivation, once the issue is resolved, Tier 5 scribe documents the resolution, and the facilitator decides whether a resolution communication should be sent down the tiers using the same method as the mitigation communication. The issue is then moved to the Resolved Workboard. The connected CIRC forms receive a signal next to their Continue to Track Workboard that an issue they escalated has been resolved. The lower tiers can review the resolution and send it to their Resolved Workboard. Resolved issues remain on the Continue to Track Workboard for a designated period before automatically moving to the Resolved Workboard. This ensures Workboards stay clear of inactive issues, reducing clutter and maintaining focus on active work. Resolved issues remain archived but can be reactivated at any time. If reactivated, the issue retains complete historical data and reappears on the Active Workboard for continued resolution. Logic forces unresolved items to remain in the Active Workboard or Queue until resolved.

Regarding sustainment reports, embodiments of CIRC further include a Sustainment Reporting module that provides organizations with automated analytics and compliance tracking to ensure that the system is being used consistently, accurately, and effectively over time. These reports address a critical barrier for many employers: sustaining adoption of high reliability practices across large, multi-tiered organizations. With regard to compliance monitoring, sustainment reports monitor key compliance measures, including: Associate Engagement, which includes tracking whether frontline associates submit required safety recognitions, “good catches,” or escalations at designated intervals (e.g., three submissions weekly, two issues monthly). Manager Engagement may include verifying whether Tier 1 managers log in daily to conduct their huddles, review escalations, and process new submissions as intended. Leadership oversight may include sending automated alerts to directors and upper tiers when managers fail to process submissions or complete daily requirements.

Regarding timeliness and accuracy metrics, the reports extend beyond participation and also measure quality of performance, including resolution timeliness, which may include comparing resolution times against predefined thresholds for each priority category (e.g., high-priority issues resolved within four hours). The reports may also include escalation accuracy which may include verifying that issues are being escalated to the correct tier based on embedded algorithms, preventing both under-escalation and unnecessary escalation. Further, the reports may include huddle completion, including confirming that unit, practice, and tiered huddles are occurring daily and that escalations are accurately discussed and documented.

With regard to communication effectiveness, sustainment reports also track the effectiveness of communication loops, including whether top-tier communications (e.g., safety alerts, time-sensitive updates) are cascaded down through the Digital Agenda and acknowledged at the frontline, which communication channels (platform alerts, email, newsletters, verbal readouts) are being utilized, and whether required messages are being delivered consistently across all practices and units.

Regarding standardization and reliability, by aggregating these data points, the Sustainment Reporting module ensures that daily tiered huddles are standardized and reliable, with every unit and practice following the same structured approach provided through the system. Drift is detected early, including practices that skip huddles, delay escalations, or leave escalations unresolved once they reach their Workboards. Accountability is enforced by reporting out unaddressed escalations and issuing targeted alerts to leadership until resolution occurs. Corrective interventions can be deployed by leadership to re-align practices, reinforce expectations, and sustain high reliability behaviors across all tiers.

Regarding visual workboards and alerts, reports may be displayed in Workboards with color-coded compliance indicators (green/yellow/red), making it easy for leaders to see areas of strength or noncompliance. Additionally, real-time alerts can be issued when thresholds are not met, ensuring swift intervention.

Regarding scalability, the Sustainment Reporting module is designed for scalability across large health systems and enterprises, capable of monitoring compliance across hundreds of ambulatory practices and multiple hospitals simultaneously. This ensures organizational leaders can maintain oversight and consistency at scale.

CIRC made this speed and coordination possible. The connected CIRC forms acted as a central brain, keeping every tier interconnected and displaying historical information from all previous levels so that leaders reviewed the same accurate, consistent record without re-entry or lost details. Its Active Workboard automatically prioritized the most critical risks, guiding facilitators to address the highest impact issues first. The system's embedded algorithms optimized both performance and reliability by ensuring decisions followed structured pathways rather than ad hoc judgment. Ultimately, this example demonstrates how CIRC transforms risk escalation from a fragmented, delayed process into a streamlined, transparent, and highly reliable system, one that protects frontline teams, accelerates leadership action, and safeguards the organization as a whole. Beyond rapid resolution, CIRC also drives long term sustainment by ensuring daily huddles, escalation reviews, and compliance practices occur consistently across all units. Its sustainment reporting and digital agenda features prevent drift, standardize operations, and make it easy for any leader to guide the process, ensuring reliability at scale across even the largest organizations.

With regard to application of CIRC to IT ticket escalation, the connected CIRC forms are the innovations that drive efficiencies in the IT Ticket Escalation workflow similarly to how they do in the Tiered Safety Huddle workflow. They act as a central brain, seamlessly linking IT support tiers, technicians, and managers so the department operates from a single source of truth. Workboards, algorithms, and communication features are powered by these forms, ensuring visibility, accountability, and speed at every step. Attributes of the Connected CIRC Form include ease of submission: Submitting an IT ticket is simple and fast. Frontline staff use a click-through intake form that takes under a minute. This encourages reporting and eliminates barriers to surfacing problems quickly. CIRC also provides real-time transparency for submitters: Once submitted, the ticket lives in a connected CIRC form. Additionally, automated systems, potentially including artificial intelligence models, may use collected metrics and/or other data to give estimated wait times. For example, the system could determine the type of ticket that's been submitted, it's priority level, number of other tickets currently open and their priorities and use historical resolution times to give an estimate. The person who entered it no longer waits in the dark. Rather, they can see updates in real time as technicians and managers work the issue. CIRC enables bi-directional escalation: Connected forms allow higher tiers to send tickets back down with instructions that instantly populate in the lower-tier form. This ensures tickets are always managed at the right level without losing context. CIRC provides guided algorithms: Embedded algorithms walk technicians through structured diagnostic and resolution steps. Because forms are connected, each update in this workflow is visible across tiers, driving consistent, reliable troubleshooting. CIRC enables communication directly from the Form: IT teams can send updates, alerts, and resolution communications right from their connected CIRC form. Notifications are automatically distributed via email and in-platform alerts, ensuring alignment across all stakeholders. CIRC enables reactivation of resolved records: Resolved tickets remain stored on the Resolved Workboard with full historical context. If the problem reoccurs, the ticket can be reactivated instantly-retaining all prior history and updates so the IT team never has to start from scratch.

Attributes of the Workboards (Powered by Connected Forms) include auto-populated and auto-organized data. Workboards are automatically populated by the connected forms. Tickets are organized by tier, priority, and follow-up dates so each IT team member knows exactly what needs attention today. CIRC enables highlighting of alerts. Updates made in one form instantly highlight across all connected Workboards until acknowledged, preventing missed changes. With regard to personalized views, each user sees their own Active Workboard while also maintaining visibility into escalations sent upward (Continue to Track) or resolved (Resolved Workboard). CIRC enables a daily workflow backbone. Because they present issues in real time and priority order, Workboards serve as the organizing backbone for daily IT huddles and ongoing ticket management.

CIRC algorithms driving excellence. CIRC provides decision-tree guidance. Algorithms embedded in the forms standardize escalation decisions, ensuring that tickets are routed and resolved consistently. Further, CIRC enables consistency across tiers. No matter who handles the ticket, the same structured pathways are followed, preventing variation and errors. CIRC also enables faster resolution. By guiding diagnostic steps and surfacing known solutions, algorithms increase first-pass resolution rates and reduce downtime.

CIRC also provides communication and analytics features, including closed-loop communication. From within the connected form, IT staff can push updates and resolution notes directly to ticket submitters, managers, or leadership. No separate system or manual update is needed. The features also include AI-Enhanced Workboards. Structured data and free-text inputs from all tickets feed into AI Workboards, which identify recurring problems, surface trends, and predict risks before they cause outages. The features also include System-Wide Accountability. Notifications, reminders, and required acknowledgments ensure ownership is clear at every step and no ticket can be prematurely closed.

By linking every technician, manager, and requester through the connected CIRC forms, and organizing them through intelligent Workboards, IT departments achieve easier ticket submission, encouraging rapid reporting, real-time transparency for both IT staff and the original submitter, seamless updates and communication directly from the connected form, consistent resolution guided by algorithms, and reactivation of closed tickets with full history intact.

With regard to application to Project Management, the connected CIRC forms act as a central brain, seamlessly linking every contributor, action, and decision across projects, so the team operates from a single source of truth. By replacing static task trackers with living, connected forms and logic-driven Workboards, CIRC transforms project management into a dynamic, transparent, and accountable system. Attributes of the Connected CIRC Form include a Single Source of Truth: Every task, decision, and escalation is captured in a connected CIRC form. As the task moves between contributors, the same form remains linked, eliminating duplicate updates, re-entry, and fragmented communication. Further, information comes to the user: Unlike static systems, where users chase updates, the connected CIRC forms push information directly to each user. Relevant updates, assignments, and highlights automatically appear on their Workboards, so nothing is missed, and no one wastes time searching. The features also include ease of contribution. Team members can update progress, confirm completion, or flag barriers directly in their form. Those updates flow instantly across all linked forms, so everyone sees the same information in real time. Hidden fields keep communication concise by showing each user only the fields relevant to their role, ensuring clarity without unnecessary detail. The features also include the ability to send back for completion. When SMEs or POCs finish their assigned action, they use the Send Back status to return the task to the Action Owner with a completion note. This keeps the workflow clean and ensures owners know when work is finished and ready for closure. The features also include communication from within the form: Updates, instructions, and completion messages are distributed directly from the connected form via automated alerts and in-platform notifications. This eliminates email chains and manual status chasing. Additionally, the feature include reactivation from Resolved Workboard: Closed tasks remain stored with complete history. If a barrier resurfaces, the form can be reactivated instantly, keeping prior context intact so the team doesn't start over.

In some embodiments, the system may be applied to project management workflows involving multiple functional departments, roles, or organizational units collaborating on a shared project or initiative. In this context, a project item may represent a deal, initiative, work package, or deliverable that progresses through project phases. Team based projects may have a main landing page and workboards along with individual team members or departments have their own workboards. The project workboards may be reviewed when a team has their team meetings. Each task listed on the project workboards may be linked to those assigned individuals or departments. The project workboards may include Active, Pipeline Queue, Active, At-Risk, Sustain workboards. The team member or department individual workboards may include Active, CTC, and complete. The system may generate role-specific forms and associated Workboards for participating departments or individual team members, where each Workboard represents a focused execution and coordination view for the responsibilities of that department. For example, participating departments may include development or origination teams responsible for sourcing and evaluating opportunities, underwriting teams responsible for financial and risk review, capital markets teams responsible for investor placement, construction or technical review teams responsible for feasibility and compliance assessment, legal teams responsible for transaction structuring and closing, and asset management teams responsible for post-completion oversight. These examples are illustrative and non-limiting. Each department may be provided with its own Workboard displaying the project items for which that department is responsible. These Workboards may support assignment of action owners, target dates, status updates, and system-generated alerts and notifications when actions are assigned, deadlines approach, conditions change, or escalation occurs within the project context. Coordination across departments may be maintained through shared, synchronized CIRC forms associated with each project item. While each department may interact with the project through its own role-specific Workboard, the underlying project item may be represented by linked forms that preserve shared contextual fields and cross-role visibility. Updates made by one department such as status changes, action assignments, escalation activity, or informational updates may be reflected across corresponding forms and Workboards according to permission-based logic. Through this shared form structure, each department maintains full line of sight into what other departments are working on within the project. In this way, shared CIRC forms act as a common coordination layer across departments, while Workboards provide focused, role-specific execution views.

Attributes of the Workboards (Powered by Connected Forms) include Auto-Populated and Auto-Organized: Workboards are automatically populated from connected forms. Tasks are sorted by owner, tier, priority, and target dates, so team members see exactly what requires action that day. The attributes also include Active Workboards as Living Project Boards: Each row represents a task or escalation, linked to its owner, required action, and current status. Updates remain highlighted until acknowledged, ensuring no progress is overlooked. The attributes also include Visibility Across Tiers: Contributors, managers, and executives see the same project record in real time. Leaders gain both high-level progress views and the ability to drill into frontline details, without waiting for separate status reports.

CIRC also enables a Critical Daily Workflow. Because they reflect live priorities and deadlines, Workboards become the organizing backbone of project meetings and huddles. Algorithms drive project excellence. CIRC provides Decision-Tree Guidance. Algorithms embedded in the forms guide project leads on whether a barrier should remain at the team level or escalate upward. CIRC provides Clarity on Dependencies and Risks. Logic prompts help identify next steps, interdependencies, and potential risks before they create bottlenecks. CIRC also provides Consistency Across Projects. By applying the same structured pathways to every project, CIRC ensures repeatable excellence and reduces variability in project execution.

CIRC also provides communication and analytics features, as follows. One feature is no more chasing information: Updates flow directly from the connected form to the user through automated alerts and Workboard highlights. Team members do not have to dig for updates. Instead, the information comes to them. Another feature is Concise Information Sharing: Fields hidden by role keep communication focused, reducing noise and ambiguity across forms. Another feature is Structured Data Capture: Click-through workflows reduce free-text ambiguity, turning updates into reliable, analyzable data. Yet another feature is AI-Enhanced Workboards: Structured data and free-text notes from all connected forms feed into AI Workboards, which identify recurring barriers, highlight bottlenecks, and predict future resource needs.

By linking every contributor and leader through the connected CIRC forms, which push the correct information to the user and hide irrelevant fields for concise communication, and by organizing them on intelligent Workboards, project teams gain: seamless updates and communication directly from the form, real-time visibility into both progress and barriers, consistent escalation decisions guided by algorithms, accountability through alerts, target dates, and acknowledgments, the ability to reactivate closed items with full historical context. The outcome is faster project execution, more transparent accountability, and predictive insights, enabling teams to deliver with efficiency, reliability, and scalability.

The CIRC system introduces a novel architecture that organizes tickets, tasks, and workflows through interconnected forms and Workboards that enable users to work independently while participating in a shared, collaborative process. CIRC helps organizations manage complex, multi-tiered workflows efficiently and transparently. It supports both linear and bi-directional escalation paths, so tickets can be escalated, sent back for review, or branched to parallel entities while preserving context and history. The system generates a role-specific form for each user assigned to a given ticket, which serves as their personal workspace. As a ticket or task moves through a workflow, the system tracks all actions, status changes, and updates, maintaining a complete history. At the same time, relevant information is automatically shared in real time with all users through dynamic Workboards, providing each user with an up-to-date view of their tickets or tasks while eliminating the need to navigate multiple pages or manually transfer data.

Overall, the CIRC system is a robust platform for structured, collaborative task and ticket management, combining independent workspaces, synchronized data sharing, and comprehensive workflow oversight. It is well suited for scenarios where multiple stakeholders, tiers, or departments must coordinate actions, track progress, and maintain transparency while retaining control over entity-specific responsibilities.

CIRC is distinct from known data transfer models. It is not an OSI Node-to-Node Transport. The OSI link-layer delivers frames/bits; CIRC synchronizes structured state across roles. CIRC is not replication. Databases replicate from master to replicas; CIRC nodes are co-equal and authoritative. CIRC is not monitoring. Conventional node-to-node charts measure byte volumes; CIRC actively synchronizes semantically meaningful state. Thus, CIRC introduces a novel application-layer synchronization model. CIRC can be summarized as distributed peer-to-peer synchronization of semantically structured forms, role-conditioned rendering logic to enforce context-appropriate presentation, closed-loop acknowledgment ensuring task-level completion.

Technical features and advantages include Parallel Form Structure with Logic-Driven Synchronization. For each ticket or task submission, the system automatically generates a set of linked CIRC forms—one for each entity involved. While all forms share a common structure, each can include role- or tier-specific fields based on organizational requirements. Each entity's form serves as its dedicated workspace for managing and interacting with the ticket. Despite being individualized, these forms and Workboards are logically linked to ensure that shared fields remain synchronized across all forms and visible on Workboards in real time. This is achieved through two core system behaviors: (1) Auto-Fill on Escalation (Form to Form Synchronization) and (2) Real-Time Workboard Updates (Form to Workboard Synchronization).

Regarding Auto-Fill on Escalation (Form to Form Synchronization): When a ticket is escalated to another entity, the system auto-populates shared fields on the receiving entity's form using the responses provided by the previous entity. This occurs only once at the time the new form is first opened, ensuring any updates made by the receiving entity to their form are not overwritten.

Shared Field Auto-Fill Logic is described herein. For each shared field in the receiving form, the system performs an initial check to determine whether a shared field on the receiving form is empty. If the field is empty-Populate the field with the corresponding value from the previous entity's form. If the field already has a value, the system retains the existing value and does not overwrite, while notifying the user of the corresponding value from the previous entity's form.

The system repeats this check-and-fill process for each shared field present on the form. This logic is executed only once, at the time the receiving form is first opened after escalation. Any subsequent changes to the previous form do not change the receiving form, but the receiving form may display updates from previous issue owners. This may be advantageous because different tiers may be taking separate actions to contain the risk based on department-level or system-level impact. For example, Tier 1 may require 24 hours to remove a defective supply locally, while Tier 3 may require 48 hours to address removal across all hospitals. By allowing one form to change a field without automatically changing other forms in this manner, all tiers may actively work on the issue within their respective scope at the same time.

Key Principles are set forth below. Data Integrity: Auto-fill is performed once per form instance to avoid overwriting user-entered data on the receiving form. Transparency: Ensures relevant information is carried forward for continuity and efficiency, while allowing the receiving entity to edit as needed.

In an Example Scenario, a shared field, Category, exists across forms. Entity 1 sets Category=“Supplies”. Escalation: Ticket is escalated to Entity 2. When Entity 2 opens the form: If Category is empty, it is auto-filled with “Supplies”. If Category already has a value, the value is preserved.

Regarding Real-Time Workboard Updates (Form to Workboard Synchronization): Workboards display the most current response for each shared field, based on the current owner of the ticket. As tickets move up or down between entities, the Workboard dynamically updates to reflect only the active owner's input-regardless of any background edits made by others. Ownership Determination Logic may operate as follows. The system evaluates ownership in real time using specific form-level fields. Escalation: Indicates whether the ticket was escalated to another entity. Send-back Indicators: Indicate whether the ticket was sent back to a previous entity. Status: Tracks the current state of the ticket (e.g., currently fixing, sending back, resolved). Notably, Active workboards may be configured to always show the responses from their respective tiers, while other workboards show the most current responses across tiers as described above. For example, Tier 1 marks issue as high priority and continues to work on it at their tier while escalating to tier 2. Tier 2 opens the form and determines the issue to be medium priority. Tier 1 active workboard shows priority as high, Tier 2 active workboard shows priority as medium, if tier 2 escalates to tier 3, tier 3 form will auto-fill with medium priority, if tier 1 moves the issue to Continue to Track, the priority would display as medium on the workboard (if they open their tier 1 form, it would still show as high).

Ownership logic uses the following rules: If the ticket is escalated, and not sent back, and not resolved, then the current owner=the higher entity. If the ticket is escalated, and resolved, then the current owner=“Resolved at/by [that entity]”. If the issue is escalated, and sent back to a previous entity, then the current owner=Previous Entity. If the ticket has not been escalated, then the current owner=Original Entity The system evaluates this logic to all forms associated with the ticket to determine the current owner.

With regard to Workboard Behavior, once the current owner is identified, the Workboard will: display the most recent responses from the current owner's form for all shared fields, ignore edits by non-owning entities, even if those entities are actively editing their forms, and update dynamically whenever ownership changes.

Key Principles include Dynamic Ownership: Ownership is continuously updated as the ticket moves between entities. The key principles also include Single Source of Truth: The Workboard reflects shared field values only from the entity currently responsible for the ticket. If there are discrepancies between tiers, both tiers' responses are displayed and the current/highest tier's response is the final response used in metrics. Further, the principles include Non-Interference: Edits made by non-owning entities do not affect the displayed Workboard values. Another principle is Visibility of Resolution: If a ticket is marked resolved by the current owner, that resolution state is reflected in ownership.

In an example scenario, in an Initial Assignment, Current Owner=Entity 1 and Workboards display Entity 1's responses for shared fields. The issue is Escalated to Entity 2: The Current Owner=Entity 2 and Workboards display Entity 2's responses for shared fields. The issue is Escalated to Entity 3: The Current Owner=Entity 3 and Workboards display Entity 3's responses for shared fields. The issue is Sent back to Entity 2. The Current Owner=Entity 2 and Workboards display Entity 2's responses for shared fields. Entity 2 resolves the ticket. The Current Owner=‘Resolved by Entity 2’ and Workboards display Entity 2's responses for shared fields

Together, these two mechanisms, form-to-form synchronization and form-to-Workboard synchronization, ensure that all users have consistent, up to date visibility into ticket activity, while preserving the individuality and data integrity of each entity's workspace. Currently available platforms provide the options to work in a single shared document, or in individual, siloed documents. The individual, logically linked, CIRC forms provide the best of both models: the transparency and collaboration of a shared document, alongside the privacy and flexibility of individualized forms tailored to each user's role and workflow.

This feature provides the following technical advantages: Real-Time Collaboration: All users can see shared ticket data as it is updated; Configured Workflows: Each user's form can contain fields tailored to their responsibilities without cluttering the interface for other CIRC forms; Privacy and Compliance: Individual CIRC forms can allow sensitive fields (private notes, PHI, etc.) to remain visible to designated roles only and be excluded from data syncing across CIRC forms and Workboards; and Traceability: Because each user interacts with their own CIRC form, the system can log activity and changes for each user on every ticket.

CIRC enables transparency across escalation layers/user roles. A key technical feature of the CIRC platform is its system-enforced transparency, which ensures that all users involved in the lifecycle of a submission, regardless of role, tier, or escalation level, have appropriate visibility into the ticket's status, history, updates, and outcomes. This transparency is governed by role-based access controls, ensuring that users can view relevant information without breaching data boundaries or visibility rules specific to their role or entity. This is accomplished through a combination of Dynamic Form Headers with Historical Context. Each user's CIRC form includes a dynamic header section that is automatically populated with key information submitted by prior entities in the escalation chain. This ensures that when a ticket reaches a new entity, all relevant context and previous updates are immediately visible in one centralized location. This design improves workflow efficiency, reduces duplication, and enables informed decision-making without requiring users to reference other forms. Each time a new entity begins working on a ticket, the system generates a new section within the summary table across all associated forms. This table functions as a structured, cumulative log of the ticket's progression. Each section of the summary table is populated through field-level data mirroring. Each designated field within the summary table references and displays the corresponding value entered in the previous entity's form. This creates a real-time, unidirectional linkage between source fields (on the prior entity's form) and target display fields (in the summary section across all forms).

As previous entities update their forms, those updates are immediately reflected in the summary table, regardless of current ticket ownership. The system performs this mirroring across all forms tied to the ticket. This means that the Workboard always reflects the most up-to-date responses from the current owner and the summary table on each form provides a complete historical view of all inputs submitted by each entity throughout the lifecycle of the ticket. This approach ensures that all users have immediate access to both the current state and the full progression of a ticket without having to navigate through multiple interfaces or override prior entries.

CIRC enables Dynamic user-specific Workboards. CIRC includes dynamic Workboards that are automatically generated and updated by the system as new information is entered by any user. Each Workboard row represents an individual ticket or task and includes a direct link to its corresponding CIRC form. Workboard columns provide a real-time, high-level overview of status, updates, escalation state, and assignment. This user and role specific Workboard structure improves operational efficiency, eliminates confusion regarding task ownership, and streamlines visibility into both current workload and historical context.

CIRC provides an Active Workboard. The Active Workboard displays all tickets or tasks currently assigned to a user for action. Items appear here by default when first assigned, provided no follow-up date has been entered. This Workboard serves as the user's primary workspace for in-progress work requiring immediate attention. An item is on the Active Workboard if no target date is entered, the status is not resolved, and it either has not been escalated or it has been sent back. All of the following must be true: the target date field is blank, and the status is not ‘Resolved’, and either the item has not yet been escalated to the next entity, or the item was sent back from the next entity

CIRC provides a Queue Workboard. The Queue is a subset of the Active Workboard. It displays all assigned tickets/tasks that include a designated target or follow-up date. Items in the Queue remain active but are organized chronologically for long-term tracking. This structure helps maintain a clean, actionable Active Workboard, separating urgent items from long-term or follow-up tasks without losing visibility. An item is on the Queue if a target date is entered, the status is not resolved, and it either has not been escalated or it has been sent back. All of the following must be true: The target date field is NOT null, and the status is not ‘Resolved’, and either the item has not yet been escalated to the next entity or the item was sent back from the next entity

CIRC provides a Continue to Track workboard. The Continue to Track Workboard displays issues/tasks that the user previously handled (e.g. submitted, reviewed) but which have since been escalated or reassigned. This feature ensures continued visibility into outcomes and progress, even after the ticket leaves the user's direct ownership. It promotes transparency and accountability by allowing users to follow the lifecycle of issues they were previously involved in. The Continue to Track Workboard automatically updates as other users make changes to the ticket, including new entries, status updates, or final resolutions. When a ticket on the Continue to Track Workboard is resolved by another entity, the ticket will remain visible on the Continue to Track Workboard for ongoing reference; automatically move to the Resolved Workboard after a defined period (default: 2 weeks; configurable); and be manually archived earlier by the user if desired. Additionally, when an item on a Continue to Track workboard has been resolved, a notification will appear on the Active Workboard notifying the user that an item they have been monitoring is resolved. They can click into the issue to review resolution notes and move it to their Resolved Workboard. An item is on the Continue to Track Workboard if it has been escalated and not sent back, the status is not “resolved” or the status is “resolved” and the date of resolution is less than two weeks ago, and it is marked as ‘Continue to Track’.

The Continue-to-Track Workboard may be used after a clean handoff, when the originating tier has relinquished responsibility for execution and resolution. In this state, the originating tier no longer owns the work, has no assigned actions or target dates, and is not responsible for solving the issue. However, Continue-to-Track is not a read-only state. While the originating tier is no longer executing remediation, it may still provide informational updates to ensure shared awareness and accuracy. For example, if conditions change or new relevant information becomes available, the originating tier can update the issue so that the tier responsible for resolution has the most current context. These updates are informational in nature and do not represent ownership, execution, or corrective action. The purpose of Continue-to-Track is to support visibility, communication, and situational awareness after a handoff, while preserving a clear separation between monitoring and execution. By contrast, when a tier is actively performing remediation while escalating for broader coordination, the issue remains on the Active Workboard. Continue-to-Track is used only when execution responsibility has been fully transferred. Regarding a workflow of multiple tiers actively working on a concern at their level while escalation is occurring: When a tier escalates an issue while still fixing it, the issue remains on the Active Workboard. When the user opens their form, it may display linked upper-tier forms to provide line-of-sight visibility into what is happening at higher tiers. As long as the status remains “fixing,” the issue stays on the Active Workboard even while escalated.

CIRC also provides a Resolved Workboard. The Resolved Workboard displays all tickets or tasks that have been officially marked as resolved. These records are retained to support historical reference, auditability, and reporting. Users retain access to resolved items and may, if needed, manually re-activate a resolved ticket. Upon reactivation, the item will return to the Active Workboard, allowing further updates or follow-up actions, or escalation. An item is on the Resolved Workboard if it has been escalated and ‘send to Resolved Workboard’ is selected and has not been sent back, or the status is resolved, and ‘send to Resolved Workboard’ is selected or the date of resolution is more than two weeks ago. Any of the following primary conditions must be true. In Condition A: Item does not need tracked, the item has been escalated to the next entity and the item has not been sent back from the next entity and the item has been marked to ‘send to Resolved Workboard’. Condition B: The item is resolved, the status is ‘Resolved’, and either the item has been marked to ‘send to Resolved Workboard’ or the difference between Date of Resolution and ‘Today’ is more than 14 days.

CIRC provides a Shared Project Workboard (Project Management Specific). The Shared Project Workboard provides a centralized, real-time view of all tasks, updates, and progress associated with a specific project. It is accessible to all users assigned to that project, enabling team-wide visibility without requiring manual coordination or status updates. Users can seamlessly transition between: the Shared Project Workboard, showing all project-related tasks across the team and their user-specific Workboards, displaying only tasks assigned to them personally.

This interconnected structure ensures that individual users remain focused on their responsibilities, while still having full visibility into broader project activity and team contributions. The Shared Project Workboard is automatically synchronized with all related user-specific Workboards within project-management CIRC implementations. As users update their forms, the shared view reflects those changes in real time, eliminating the need for duplicative tracking or separate project reporting tools.

CIRC enables Automated resolution communications. The CIRC platform includes an automated communication mechanism that ensures timely and comprehensive communication of final outcomes or resolutions throughout the entire escalation chain, and optionally to other relevant departments, service lines, or stakeholders. This feature is designed to promote organizational transparency, maintain awareness, and formally close the loop on each ticket or task. CIRC provides resolution detection. When a ticket or task is marked as resolved within the system, based on the status fields on the form associated with the current owner, an automated communication workflow is triggered. CIRC also performs Identification of Recipients. The system identifies all prior contributors involved in the lifecycle of the ticket. This includes every entity that previously owned or interacted with the ticket through escalations, send-backs, or any form submissions. Additionally, the system can be configured to include other designated departments, service lines, or roles outside of the immediate ticket chain, ensuring wider organizational visibility as needed. The entity that resolves the ticket may select to include any of these options in the communication. With regard to Communication Content, the message sent to recipients contains: A summary of the final resolution or outcome provided by the entity that resolves the ticket, key data fields from the ticket, including relevant shared fields and any resolution notes, historical context and timeline, showing the progression and escalation path, and links to view the full ticket and related CIRC forms in the system. Regarding the Delivery Mechanism, Communications are sent as email notifications, in-system alerts, or integration with existing organization communication tools and notification method(s) may be selected by the entity resolving the ticket or by recipient roles and preferences, ensuring appropriate and concise messaging.

This feature of transparency provides the following technical advantages: Automated Visibility: Workboards and form headers are automatically populated and updated based on system logic and organizational configuration, eliminating the need for manual searching and tracking. Users who escalate or assign tickets/tasks can monitor all updates made by other team members in real time-even after ownership has transferred; Collaborative Project Oversight: For projects involving multiple users, the Shared Project Workboard provides a unified view of team assignments and progress; Easy Historical Access: Past tickets can be quickly located by searching ticket numbers, keywords, or involved users, simplifying audit and review processes; Reduction in Redundant Work: The Continue to Track Workboard enables users to follow ticket progress after escalation, reducing duplicate inquiries and re-submissions; Enhanced Accountability: All participants remain informed of outcomes and updates in real time, ensuring responsibility and follow-through throughout the ticket lifecycle.

By dynamically presenting personalized Workboards tailored to each user's role and involvement, the system ensures transparency is proactive, continuous, and enforceable rather than reliant on manual searches by the user or discretionary communication and updates between entities.

CIRC utilizes a Logic Based Guidance Algorithm for Ticket Owners. The CIRC system includes a configurable user guidance algorithm designed to assist users in determining the most appropriate course of action during ticket or task review. This algorithm evaluates user inputs in real time and applies predefined decision-tree logic to suggest specific next steps, improving consistency and decision-making across the platform. When an entity is assigned a ticket, the system presents a structured series of questions that assess key factors such as the nature and severity of the issue, its prevalence and/or frequency, and ability and resources available to resolve the issue locally. Based on responses to these questions, the algorithm analyzes the input and provides real-time recommendations, such as: “You may be able to resolve this issue without escalation. See recommended steps below” or “This issue meets criteria for escalation. Please select the appropriate escalation path.” These recommendations are automatically generated using logic defined by the organization during app configuration. During system setup, organizations define the list of decision questions, valid response options for each, and the logic rules mapping response combinations to recommended actions. This logic is fully configurable to support various use cases such as issue escalation, reassignment, resolution at the current level, or send-back to a previous handler. Users are able to override this suggested course of action, however the built in logic and user prompts promote uniformity and consistency in how users across the organization escalate, reassign, or resolve tickets. This feature of guidance algorithms for ticket owners provides the technical advantages. The advantages include Real-Time Decision Support: The algorithm evaluates user inputs instantly, allowing users to receive guidance without delay, improving efficiency during ticket or task review. Users receive context-sensitive suggestions that streamline decision-making, reducing cognitive load and training requirements. The technical advantages also include Consistency Across Users and Reduced Human Error: By applying standardized decision-tree logic, the system ensures that all users follow the same rules and procedures, reducing variability in task handling. Guidance based on predefined logic helps prevent missed steps or incorrect actions, enhancing data integrity and process reliability.

CIRC enables Workload Reduction and Enhanced Analytics via Discrete Data Collection. The CIRC system is built to prioritize structured, discrete data collection over free-text input wherever possible. This design supports intelligent automation, reduces manual workload, and enables comprehensive analytics. Discrete fields, such as radio buttons, dropdowns, and checkboxes, are leveraged to drive dynamic interface behavior and real-time end user guidance. The operations include Category Selection. When an end user initiates a ticket or issue report, they select from predefined categories (e.g., “Email Setup>Add Shared Mailbox”). Each selection is mapped to a backend decision tree to determine available self-service guidance. The operations also include Self-Resolution Guidance: If matching guidance exists, the system displays resolution assistance inline within the same interface (e.g., step-by-step instructions, screenshots, or short videos). The user is prompted to attempt resolution before proceeding with ticket submission. The operations also include Resolution Confirmation: The system asks: “Was the issue resolved?” If yes: Ticket process is terminated and logged as self-resolved. If no: The form remains populated with previously entered data, and the user continues the submission process uninterrupted. The operations also include Data Logging and Analytics: The system tracks key interactions for analytical purposes, including: Number of tickets initiated where guidance was triggered; Number of tickets submitted vs. abandoned (self-resolved), and Categories with the highest self-resolution success rates. This feature of end user assistance and discrete data collection provides the following technical advantages: Automated Routing: Submissions can be routed automatically based on selected category and severity, reducing reliance on manual triage; Uniform Data Collection: Structured data input enables downstream analytics, filtering, and reporting across organizational units; Error Reduction: Users are prevented from submitting incomplete or invalid entries due to field-level validation and restrictions; and Workflow Triggers: Specific selections can trigger tailored workflows or required escalations pathways automatically.

CIRC Reduces Workload by addressing routine issues at the source, support staff ticket volume is reduced. Additionally, CIRC enables Improved Response Efficiency: Resources can be allocated to complex or unresolved tickets rather than simple routine issues. End users can often have their issue resolved without needing to wait for assistance. With regard to analytics regarding required support level, CIRC provides an ability to track self-resolution efficacy and submission categories that require higher levels of support.

CIRC utilizes standardized backbone workboard structures. Examples of such a structure are provided below. The structures described below may be fixed at the system level, while organizations may configure parameters within defined guardrails. CIRC applies consistent coordination backbone across tiered safety huddles, team-based project management, and IT ticketing. Organizations may configure settings to fit their needs, but core workflow behaviors remain the same.

A Tiered Safety Huddles structure may provide daily identification, escalation, and resolution of safety, quality, and operational risks across organizational tiers. Standard Workboards for these huddles may include: (1) Active Huddle, issues currently being worked at the local tier. Ownership, actions, or target dates exist. Escalation does not remove items from Active. (2) Queue, issues with a future target date and on target date, auto populates to Active WB for review. (3) Continue to Track, execution responsibility has moved to another tier. The local tier is no longer acting, but visibility is retained for awareness and follow-up. Lower tiers can provide updates to upper tiers. (4) Resolved, issues addressed and closed. Outcomes are captured for reporting, learning, and trend analysis. Features of this structure may include: Escalation does not equal handoff. Issues may appear on multiple workboards simultaneously. Movement between workboards is system-driven. Tier authority governs overrides and visibility.

A Team-Based Project Management structure may provide coordination and execution of cross-functional projects and initiatives over a longer time horizon. Project phases describe the nature of work within a project item. Workboards manage attention, prioritization, and execution flow. Phases and workboards are intentionally separate. Standard Workboards for Team-Based Project Management may include: (1) Pipeline/Queue, future or planned work that is not yet execution ready. Includes items in Pipeline, Scope, Current, and Future phases, with corresponding status fields. (2) Active WB, a rolling near-term execution window (for example, the next one to two weeks). Tasks would auto populate into the Active Workboard from the Pipeline/Queue Workboard. Includes work that is imminent or currently executing. Drives action owner alerts and coordination. Escalation does not remove items from Active. (3) At-Risk/Escalation, a visibility overlay for items requiring awareness or coordination. Items may appear here in addition to Pipeline or Active. Escalation does not stop execution. (4) Sustain, the terminal attention workboard for projects. Execution is complete and/or responsibility has been reduced. This board is used to monitor impact, effectiveness, and outcomes. Completed work resides here; completion is represented as a state or flag, not a separate workboard. Features of this structure may include: Parallel execution and escalation are supported. Movement is system-driven based on readiness and timing. Active remains intentionally focused. Individual team members' views may differ from the project level views without changing system logic. Team member WB: Active, CTC, Complete

A Tiered IT Ticketing structure may provide operational management of IT incidents and requests with clear ownership, escalation, and visibility. IT ticketing mirrors the tiered safety huddle backbone with a simplified flow appropriate for continuous operational work. Standard Workboards may include: (1) Unassigned, all new tickets land here. Tickets await triage and ownership (AI can support). High-severity tickets may escalate immediately. (2) Active, tickets that are owned and being worked. Tickets are expected to be worked daily. Troubleshooting, remediation, and coordination occur here. Most likely there will be a hand off structure but can escalate and remain working on issue. (3) Continue to Track, execution responsibility has transferred (hand-off) to another tier, team, or vendor. The local team is no longer acting, but visibility is retained. (4) Resolved, the fix has been applied and the ticket is closed. Resolution details are preserved for metrics, reporting, and learning. Features of this structure may include: There is no separate queue after assignment. There is no At-Risk workboard for IT ticketing. Risk, blockage, urgency, and escalation reasons are expressed through status, priority, and escalation fields that trigger visibility and escalation without moving tickets out of Active.

All workflows within CIRC may use forms that share a standardized structural framework. This framework defines the core fields, sections, and data relationships required to support system logic, escalation, workboard behavior, reporting, and analytics. Core form elements are consistent across organizations and workflows and may include, for example: (1) Identification and classification fields (2) Ownership and role-based responsibility fields (3) Status, priority, and escalation indicators (4) Action items, target dates, and resolution details (5) Structured notes and outcome documentation. These core elements are fixed at the system level to preserve consistency, interoperability, and auditability. Within the standardized framework, organizations may configure certain aspects of the forms to reflect local preferences without altering underlying system behavior. Configurable parameters may include: (1) Category values and taxonomies used in drop-down fields; (2) Role lists and assignment options; (3) Optional field visibility by role or tier; (4) Ordering and grouping of fields within defined sections; (5) Default values or labels presented to users; Configuration is constrained to predefined options and does not permit unrestricted customization of form logic, data structures, or workflow behavior. CIRC forms are standardized by design and configurable by preference. The structure and meaning of the data captured remain consistent across deployments, while organizations can adjust presentation and selectable values within defined guardrails.

For each project or initiative, CIRC provides a project-level landing page that serves as a central location for key context and reference information. The landing page may display standardized contextual fields such as a problem description, objective, targets, or current state summary. The landing page presents context and reference information. Execution, coordination, ownership, and escalation are managed through the standardized workboards. The landing page content and layout may be configured within defined guardrails, while underlying structure and system behavior remain unchanged.

CIRC may provide the organization with a guided configuration workflow, which may allow streamlined customization of the app that does not require high level technical knowledge. Steps that may be involved in this workflow are detailed below. An example of app configuration steps may include: Select the primary purpose or use case, define the tier or department structure, CIRC Form Customization Workboard Customization, Option Reminders and Communications.

Configuration Step #1: Primary Purpose/Use Case. The organization will select the primary purpose of this set of CIRC forms and Workboards. This selection will automatically generate template CIRC forms, Workboards, and logic guided algorithms. Standard templates are provided for the following use cases, however blank CIRC and Workboard templates can be provided for other use cases: Tiered Safety Huddles, Ticket Escalation (IT, etc.), Project Management.

Configuration Step #2: Define the Tier/Department/User Structure. The organization will first define the tiers/groups/user roles that will be involved in ticket/task workflows. Next, the organization will list all users that should be assigned to each defined group. For an escalation pathway (Tier Safety Huddles, IT ticket escalation, etc.) this will typically be tiers of staff/leadership such as front line employees, managers/supervisors, department leads/senior managers, etc. For a project management pathway, this will typically be each role required for project completion. For smaller-scale projects, the project manager may determine which template to use and select which users to add to the project management team. This selection populates dropdown fields and associated workboards. When a project or tiered huddle structure is created, the system allows designated users or user groups to be associated with that project or huddle based on defined roles and access rights. These associated users represent the set of individuals eligible to be selected as action owners or points of contact within that context. When a form is later completed for that project or tiered huddle, the system automatically populates a selectable list with the names of those associated users. The user completing the form selects the appropriate action owner or point of contact from this list, and the system links that selection to the underlying user information. Upon saving the form, the system detects the assignment and automatically triggers an action owner alert.

Configuration Step #3: CIRC Form Configuration. Based on the primary purpose/use case selected in step 1 and the tiers/groups defined in step 2, the system has automatically generated template CIRC forms templates. The template can be adopted as is or configured to meet any organization specific needs. Generally this configuration is limited to organization structure (departments, tiers, etc.) and organization specific questions on the CIRC form. The logic on forms and workboards does not change.

CIRC Configuration for Escalation Pathway Template (Tiered Safety Huddles or IT Ticket Escalation). CIRC Section 1: Assessment questions to determine if the current entity is the appropriate owner. If so, the system provides guidance on next steps and resolution, and if not, the system provides guidance to escalate the ticket. Configure fields in section 1: Typically this section will be a series of yes/no questions, that may include branching logic, that are included in a pre-defined, configurable algorithm to guide the user through a structured workflow. Based on their selections, they will be advised to continue working on the escalation themselves/at their tier, or to escalate for further assistance. Fields provided on the template CIRC form include: 1. Can this issue be resolved by [ENTITY NAME]?—Yes/No. 2. Could this issue be impacting other areas?—Yes/No. The entity name will auto-populate in the CIRC form based on information previously entered in step 2 of the configuration workflow.

Configure branching logic: The template CIRC form advises users to: (1) Work on the issue/concern at themselves/at their tier if they are able to resolve it AND it is not impacting other areas; and (2) Escalate the issue/concern if they cannot resolve the issue/concern OR it could be occurring in other areas. During configuration, the organization is provided with a table to configure this decision tree logic. This table contains all fields defined in Step 1 of CIRC form customization in rows, and their available answer choices as columns. The organization is prompted to select the answers that would indicate a user should work on the issue/concern themselves/at their tier.

For example, the template CIRC form table would look like this:

TABLE 1

| Question: | Yes | No |
|---|---|---|
| Can this issue be resolved at [GROUP NAME]? | X | |
| Could this issue be impacting other areas? | | X |

The organization will then define the combination of answers required for each prompt (work on at your tier or escalate). They will be asked: “Do all questions need to be answered as described above in order for the ticket to be worked on at this tier?”. If yes, branching logic is done here. If no—How many need to be true? If one, logic is done. If more than one—select groups that need to be true, i.e. 1&2 or 3&4 need to be true. Or 1&2 or 3 need to be true.

In the template CIRC form, both questions need to be answered as described in the table in order for the user to be prompted to work on the ticket at their tier. If either question is not answered as described in the table, the user should be prompted to escalate the ticket.

CIRC Section 2: Fields to complete when working on the escalation. The built-in template contains the following fields, with the option to add, remove, or edit fields as needed. Action Owner-searchable dropdown menu containing all users. Point of Contact-searchable dropdown menu containing all users. Category-Dropdown. Priority-Dropdown. Status-Dropdown. Target/Follow-Up Date-Calendar Selection. Notes-Free text notes box. Additional fields as needed.

The organization may set up branching logic as needed within the template fields or custom fields they add. For example, an organization may choose to set up subcategories such as: Supplies, including shortage, defect, etc. In this case, they would set branching logic to show an additional subcategory dropdown if ‘Supplies’ is selected as the category.

CIRC Section 3: Escalation & Workboard Selection. Escalation Decision. The user is asked if they would like to escalate the issue/concern to the next tier. The algorithm defined in step 1 will provide guidance, however the user is given control over the decision to escalate. The system logs data to show how many tickets follow vs override the suggested workflow allowing the organization to run analyses to provide opportunities to improve workflow and guidance or increase employee education and support. Do you want to escalate to [GROUP NAME]? The answer may be yes or no. If yes, the user is provided with a searchable dropdown to select the entity the ticket should be sent to and the reason for escalation. The group name will auto-populate in the CIRC form based on information entered in step 2 of this configuration workflow. Some configurations will allow an organization to automate this process so that the algorithm makes selections without user input.

Workboard Selection. If a user selects to escalate an issue, they are prompted to select which of their Workboards the ticket should move to. If they would like to be able to quickly view continued updates regarding ticket status/resolution, they can select ‘Continue to Track’. If the ticket is not relevant to them or they do not need to see further updates, they can select ‘Send to Resolved Workboard’. The user may change their mind about a ticket at any point and move from Continue to Track to the Resolved Workboard or from the Resolved Workboard to Continue to Track. The user may also open the CIRC form from Resolved Workboard to view updates if needed in the future. If a user does not escalate a ticket, this question remains hidden, and the ticket stays on their Active Workboard (or Queue if a target/follow-up date is selected). If a user selects to escalate a ticket, this field defaults to ‘Continue to Track’ for maximum visibility, however the user can opt to send to their Resolved Workboard if updates are not necessary or can continue working at their tier and keep it active.

The form may further include a section prompting the user to select a workboard to send this ticket to. The options may be Continue to Track or Send to Resolved Workboard.

CIRC Customization for Project Management Template. For project management, three versions of the CIRC forms will be created: (1) the intake form, (2) the task submission form, and (3) the task workspace.

I. Intake CIRC Form: The project manager will provide basic project information, such as project title, and the roles and names of the individuals involved in the project on the Intake CIRC Form. An organization may add additional fields as appropriate for their needs. The project manager will then define the project, including focus area, problem statement, current state, system analysis, future state design, and annual target(s). An outline of the template intake form for project management provided by the system includes: 1. Title; 2. Roles & Name table; 3. Focus Area; 4. Problem Statement; 5. Current State (Description 1, Description 2, Add more as needed); 6. System Analysis: (pareto, 5-whys, root cause, fish bone, common cause) (Findings 1, Findings 2, Add more as needed); 7. Future state design (Future state design 1, Future state design 2, Add more as needed); 8. Annual Target; 9. Goal/Target(s) to improve (Goal/Target 1, Goal Target 2, Add more as needed); 10. Due date for each

II. Task Submission CIRC Form. The Task Submissions CIRC Form is used by the project manager to create all project tasks that need to be completed by the team. Here they will describe the required action step, the action owner, and target date.

Section 1: Task Description Header: Auto populates information provided on intake form (problem statement, focus, analysis findings, team roles & names, goal. System Failure Mechanisms-Dropdown (Technology, Policy/Protocol, Culture, Structure, Environment, Knowledge). The form also includes Action Step Description-Textbox; Action Strength-Dropdown (Strong Actions (Hard Stops, Automating manual step to remove human error), Intermediate Actions (Standardizing processes), Weak Actions (Education)).

Section 2: Algorithm Guide. This section provides a series of guided, logic-based questions, to assist the project manager in determining if the action step described meets the project objectives and is appropriately defined. The section may include the following: 1. Does this action step achieve the objective of the original intent of this focus area? Y/N; 2. Is the timeline reasonable, but aggressive? Y/N; 3. Does the action plan have a metric that can be tracked monthly? Y/N; 4. Will an improvement in the metric result in achieving the intent of the focus area? Y/N; 5. Does the target for the metric represent a significant improvement while still achievable? Y/N; 6. Additional organization specific questions as needed. If ‘No’ is selected for any of these questions, tailored guidance will display providing assistance to resolve.

III. Task Workspace CIRC Form. The task workspace CIRC form is used by the action owner of each task to document their progress on a specific task/action step. The form may include the following: 1. Status—Dropdown; 2. Checklist listing steps required for task completion—Checkboxes; 3. Point of Contact—Textbox; 4. Ticket Number—Textbox; 5. Ticket Status—Dropdown; 6. Notes/Updates—Textbox (Assign & Communicate—Dropdown & Textbox. Users can select any other team member from a dropdown menu to assign a specific step to. After selecting a name, a textbox appears allowing the users to enter a custom message to the team member. The team member will then receive a notification with this message and a link to the CIRC form); 7. Barriers to progress-Dropdown with ‘other’ option.

Configuration Step #4: Configure Workboards. Workboard Customization for Escalation Pathway (Tiered Safety Huddles or IT Ticket Escalation) may include the following. Configure Workboard Structure, Contents, and Access. The system automatically generates custom Workboards for each group defined in step 2 of the configuration process. The organization may configure the data points shown on each Workboard and the default sorting method (highest priority—lowest priority, newest—oldest, etc.). The organization may also configure who has access to each Workboard—by default, all users within a specific group have access to that group's Workboard. After each group level Workboard is set up, the organization is prompted to select if each user should have their own Workboard, or all users in a group to use the same Workboard. If you select to have each user have their own Workboard, Workboards for each user in the specified group will auto-generate to match the group level Workboard. Configure Ticket Flow Between Workboards. Set up logic that determines whether a ticket should appear on each given Workboard. Standard logic is pre-set: Active: Shows all issues/tasks currently assigned to the user for action, without a follow-up date entered. All new tickets sent to a specific entity begin on the Active Workboard. Tickets remain on this Workboard until a follow-up date is entered, they are escalated/re-assigned, or the status is marked as ‘resolved’. Queue: The Queue is a subcategory of the Active Workboard that holds all active issues/tasks that have a target or follow-up date assigned. Continue to Track: Shows all tickets a given entity has escalated/re-assigned and marked ‘Continue to Track’ when removing from their Active Workboard. Tickets remain on an entity's Continue to Track Workboard until they are resolved. After resolution, the entity can manually move the ticket to their Resolved Workboard, or it will automatically transfer to the Resolved Workboard two weeks (configurable) after the date of resolution. Resolved: Shows tickets that have been resolved and tickets that the entity has escalated/re-assigned and marked ‘Send to Resolved Workboard’ when removing them from their Active Workboard.

Configuration Step #5: Optional Action Owner Reminders and Resolution Communications. These reminders ensure issues/tasks are not stalled or unintentionally delayed due to lack of oversight or failure to escalate when needed. Date-based reminders help ensure timely follow-up and enhance accountability for time-sensitive issues while inactivity-based reminders can serve as a ‘safety net’ to prevent unresolved issues from falling through the cracks. Action Owner Reminders Based on Target/Follow-Up Date. The system provides an optional built-in reminder mechanism that monitors issues/tasks that have target/follow-up dates assigned to send alerts to action owners as the date approaches. Reminders can be sent when a target/follow-up date is approaching, on the target date, and/or after the target date has passed. The organization can configure who receives the reminder, the delivery method (push notification, email, text, in-app alert, etc.), the reminder frequency, and the content of the message. Typically the reminder will include a link to the CIRC form for easy access to provide updates for the given issue/task and a summary of the issue/task. Reminders will be cancelled if an issue/task is moved out of the queue due to escalation, status change, and/or target date change.

Inactivity Reminders: The system provides an optional built-in reminder mechanism that monitors issue/task progression that can send alerts to issue/task owners if the issue/task remains stagnant for a defined period of time. The alert can be triggered based on inactivity within: status, user notes, escalation/ownership, etc. The organization can configure: 1. The inactivity duration required to send issue/task owner a reminder; 2. Reminder recipients: issue/task owner, management, group inbox, etc.; 3. Reminder frequency; 4. Content provided in the reminder. Typically this will include: A link to the CIRC form for easy access to provide updates for the given issue/task; A summary of the issue/task; and a Timestamp of last activity

A built-in communication feature helps ensure visibility and transparency, minimizes repeat inquiries, and fosters an environment of trust and accountability. Resolution Communication. The system provides an optional built-in communication platform that allows users to notify relevant groups when a ticket is resolved. This communication helps ensure visibility and transparency, minimizes repeat inquiries, and fosters an environment of trust and accountability. Resolution communications can be sent to both required and optional recipients. Required recipients are defined at the system level and automatically receive notifications when a ticket is resolved. These typically include the original submitter, team members that contributed to the ticket, and system specific roles, such as team leads, department leads, etc. Optional recipients may be selected by the user during the resolution process. These could include specific service lines or departments, HR, legal, etc. When a user marks a ticket as resolved, they will be provided with a ‘resolution description’ notes box that allows them to enter the communication that will be included in the notification sent to required recipients. They will also be provided with a checkbox list of optional recipients defined by the system. They may choose to send a configured message, rather than the general resolution description, to each optional recipient as needed.

The organization can configure the contents of the communication. The standard template includes: 1. A summary of the original issue/task; 2. The resolution description provided by the user who resolved the issue/task; 3. Timestamp of resolution. The organization can configure who receives the reminder, the delivery method (Push notification, Email, SMS text, In-app alert). The resolution communication feature ensures consistent closure communication, especially to the original submitter, reduces information silos by relying on manual communication, and enables a log of communication history for auditing and monitoring. Communications may also provide follow-up actions, such as safety alerts, lessons learned, etc.

Mitigation Action Communication. The system provides an optional built-in communication platform that allows notifications to relevant groups regarding immediate action/mitigation(s) required prior to resolution. At any stage in, a user owning an issue may decide to send a pre-resolution communication. When selected, the user is provided with: 1. Searchable dropdown fields to select recipients of their choice; and 2. A notes box to provide description of required actions. Free text fields may provide communication drafting assistance, e.g., by an artificial intelligence model. The organization can configure who receives the reminder, the delivery method (Push notification, Email, SMS text, In-app alert). The pre-resolution mitigation communications allow immediate feedback to the original submitter and/or relevant departments/service lines if a quick resolution is not possible.

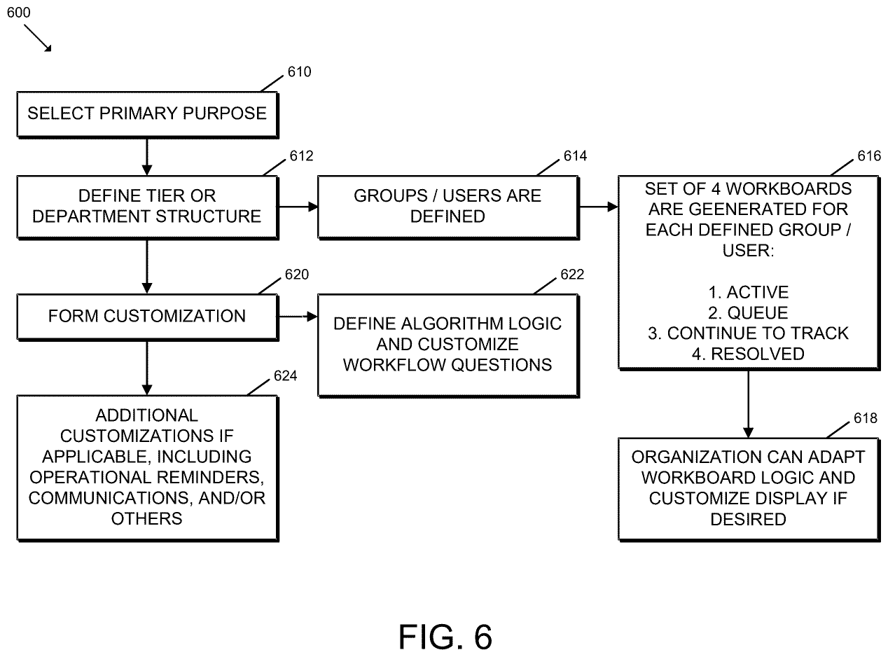

FIG. 6 represents a flow of operations 600 for customization that may be performed in connection with the system 100. In block 610, a user selects a primary purpose. In the block 612, the user defines a tier or department structure. In block 614, the user defines groups/users. Further, in block 616, the system 100 generates a set of four workboards for each defined group/user. Those standard workboards are 1. Active, 2. Queue, 3. Continue to track, and 4. Resolved. As indicated in block 618, an organization may adapt the logic underlying a workboard and configure the data displayed in a workboard, as desired. In block 620, a user may perform form customization. In block 622, the user may define algorithm logic and configure workflow questions. In block 624, the user may apply other customization, such as operational reminders, communication, and/or others.

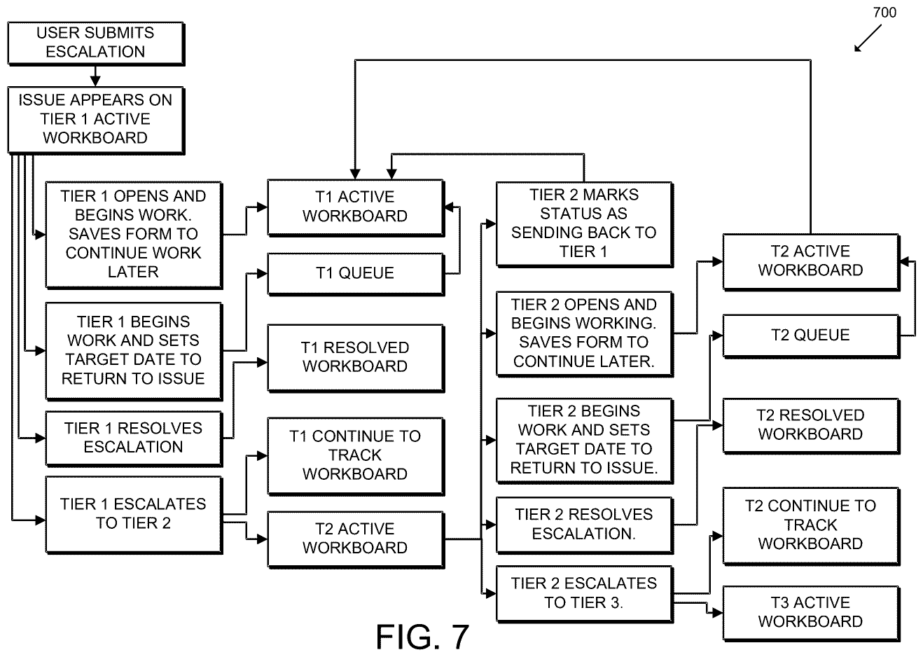

FIG. 7 represents a diagram 700 of movement of an issue through a set of workboards. For illustrative purposes, the diagram 700 is limited to a relatively small number of tiers working sequentially. Initially, a user submits an escalation. Subsequently, the issue appears on a Tier 1 active workboard. From the Tier 1 active workboard, a member of Tier 1 may perform a variety of actions. As indicated, Tier 1 may open and begin work, saving the form to continue later. In such a case, the form remains on the Tier 1 active workboard. Tier 1 may begin work and set a target date to return to the issue represented in the form. In that case, the form appears in a queue workboard for Tier 1, but is moved to the active workboard for Tier 1 on the due date. Tier 1 may resolve the escalation, in which case the issue is moved to the Tier 1 resolve workboard. Otherwise, Tier 1 may escalate the issue to Tier 2. As a result, the issue appears in the Tier 2 active workboard. Additionally, the issue may appear in the Tier 1 continue to track workboard.

From the Tier 2 active workboard, a member of Tier 2 may mark the status of the issue as sending back to Tier 1. Tier 2 may open and begin working on the form and save the form to continue working on it later. In that case, the issue remains on the Tier 2 active workboard. Tier 2 may begin work on the form and set a target date to return to the form. In that case, the issue appears on the Tier 2 queue, but is moved to the Tier 2 active workboard on the target date. Tier 2 may resolve the escalation, in which case the issue moves to the Tier 2 resolved workboard. Alternatively, Tier 2 may escalate the issue to Tier 3. In that case, the issue appears on the Tier 3 active workboard. Further, the issue may appear on the Tier 2 continue to track workboard. As indicated, an issue can be de-escalated to a lower tier. For example, an issue represented on the Tier 2 active workboard can be de-escalated, in which case the issue appears on the Tier 1 active workboard.

In certain embodiments, multiple Tiers may work on an issue concurrently. Tier 1 may both begin work on an issue and escalate the issue to Tier 2. The issue may then exist in both the Tier 1 active workboard and in one or more Tier 2 workboards. Further Tiers may work concurrently on an issue in a similar manner.

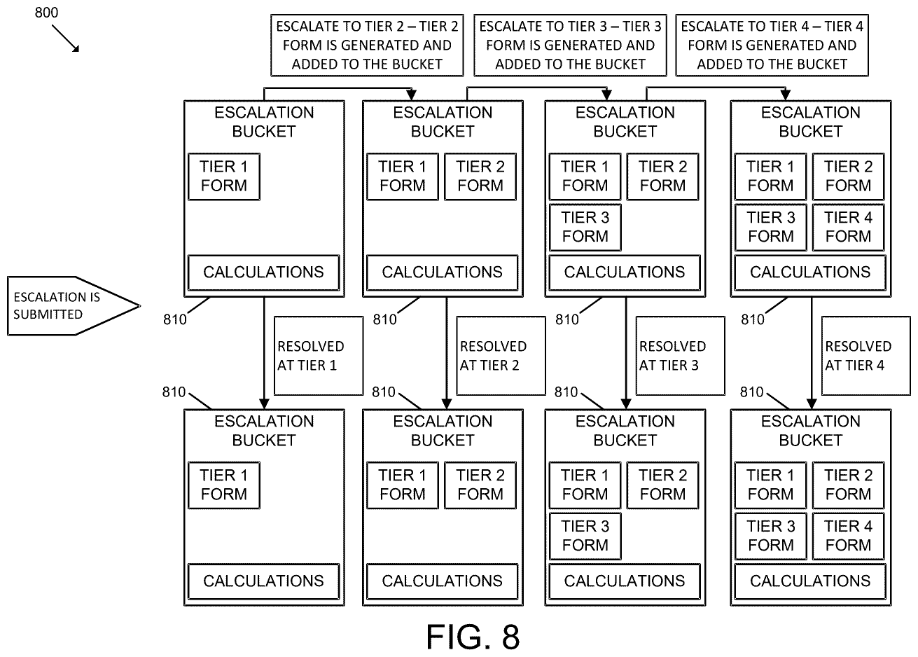

Referring now to FIG. 8, a diagram 800 represents various states of a data set 810, referred to as an “escalation bucket”, depending on actions performed within the CIRC framework (e.g., the system 100). Initially, in response to escalation of an issue, the escalation bucket 810 stores a tier 1 version of a form associated with the escalated issue. If the issue is escalated to tier 2, the escalation bucket 810 stores both the tier 1 version of the form and a tier 2 version of the form associated with the same issue. If the issue is escalated again, the escalation bucket 810 stores the tier 1 version of the form, the tier 2 version of the form, and a tier 3 version of the form. If the issue is escalated yet again, the escalation bucket 810 stores the tier 1, tier 2, and tier 3 versions of the form, along with a tier 4 version of the form.

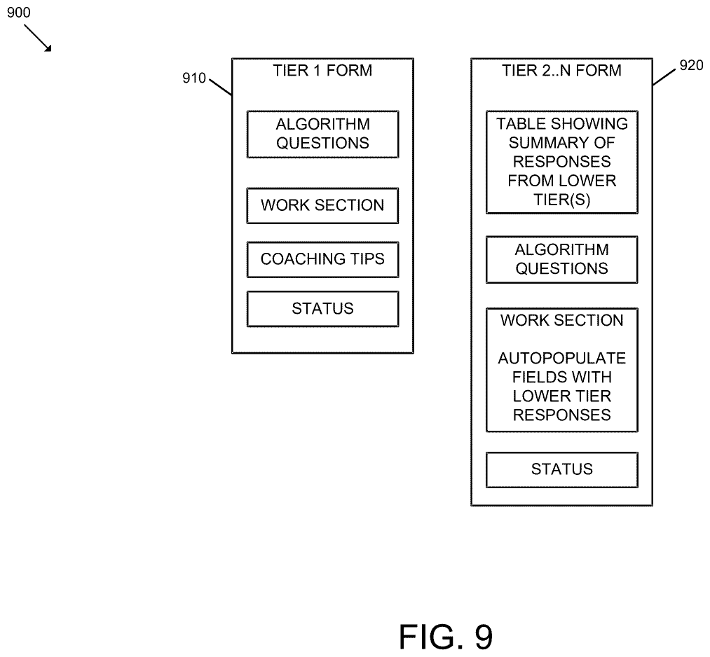

Referring to FIG. 9, a diagram 900 represents sections of a form utilized by the CIRC framework (e.g., the system 100). For a tier 1 version 910 of a form, the sections include algorithm-based questions, a work section, and a status section. A version 920 of the form for all higher tiers includes a section with a table showing a summary of responses from lower tier(s), a section with algorithm-based questions, a work section, and a status section. The work section in the version 920 is populated with responses from the previous tier. However if the present tier updates that data, the new data entered by the present tier is stored in association with that version of the form. For example, in the version 920 of the form for tier 3, the system 100 populates fields of the work section with responses that were entered in the work section of the tier 2 version of the form. However, if tier 3 changes any of that information, the system 100 stores that new data in association with the tier 3 version of the form.

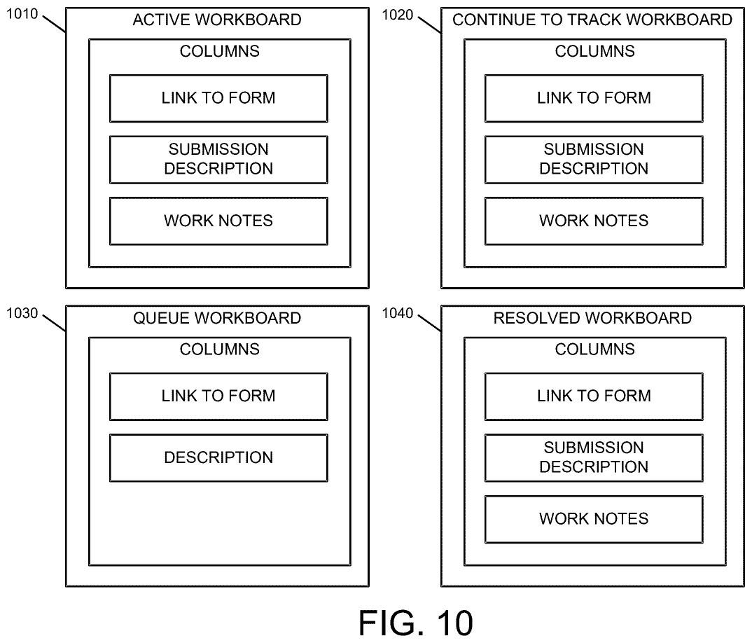

FIG. 10 represents a diagram 1000 of fields of data typically shown by each of four types of workboards 1010, 1020, 1030, 1040. In the illustrative embodiment, each field is represented visually by a corresponding column in a table. In some embodiments, one or more of the fields may be represented visually by separate columns, and/or one or more of the fields may be represented in a single column.

The active workboard 1010 includes a link to the underlying form, a submission description, and work notes. The submission description includes details from the initial submission by the end user/front line associate. Typically, that information may include the date and time of submission, name, unit/department, and type of submission. The work notes include an overview of information entered on the present tier's form. Some embodiments may include one or more of location, owner, subject title, escalation type with description, priority, and/or notes from other tiers.

The continue to track workboard 1020 includes a link to the underlying form, a submission description, and work notes. The link to the underlying form and the submission description are similar to those described above relative to the active workboard 1010. The work notes for the continue to track workboard 1020 include an identifier of the current owner and an overview of information entered by the current owner. Some embodiments may include one or more of location, owner, subject title, escalation type with description, priority, current tier, action owner, target date, resolution date with description, and/or notes from other tiers.

The queue workboard 1030 includes a link to the underlying form and a description. The description includes a brief overview of the present tier's notes and the target date (e.g., due date) that has been defined for the form. Some embodiments may include one or more of subject title, action owner, and/or target date.

The resolved workboard 1040 includes a link to the underlying form, a submission description, and work notes. The submission description is similar to that described above in connection to the active workboard 1010. The work notes include the tier that resolved the issue, an overview of information provided by the resolving tier, and abbreviated notes from all tiers. Some embodiments may include one or more of location, escalation type with description, resolution date and description, and/or current tier.

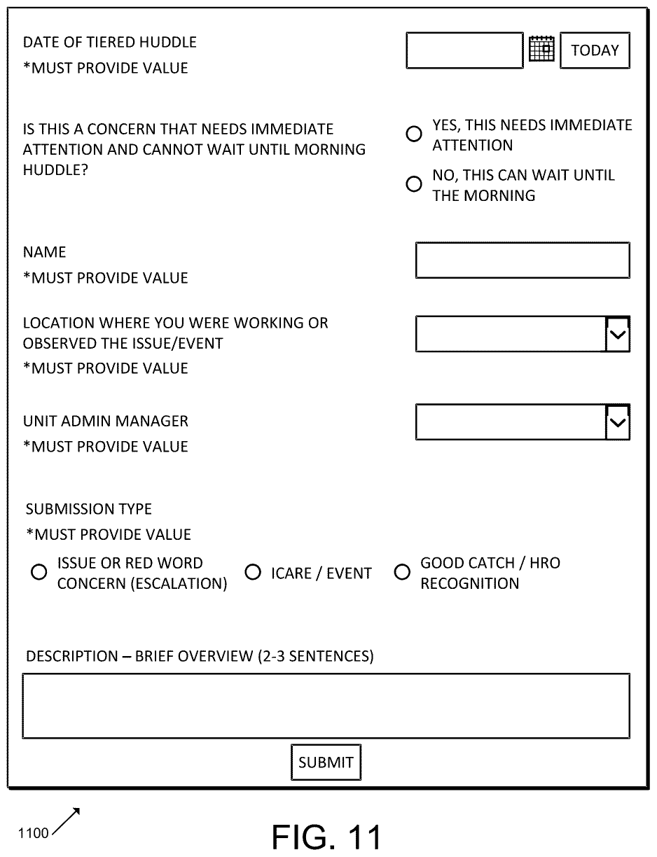

FIGS. 11-19 represent user interfaces (e.g., forms and workboards) that may be presented by the system 100 in connection with a flow of operations. Referring now to FIG. 11, a user (e.g., a front line associate) observes an issue and utilizes a form 1100 to report the issue. As shown, the form 1100 prompts the user to specify the date that the issue was observed, whether the issue needs immediate attention, the name of the user, where the user observed the issue, the user's administrative manager, the type of the submission, and a brief description of the issue. Based on logic underlying the form 1100, the system 100 may adjust the questions asked on the form as a function of responses provided by the user, such as by selectively hiding or revealing questions and corresponding user interface elements (e.g., radio buttons, drop downs, text boxes, etc.) to enable the user to answer the corresponding questions.

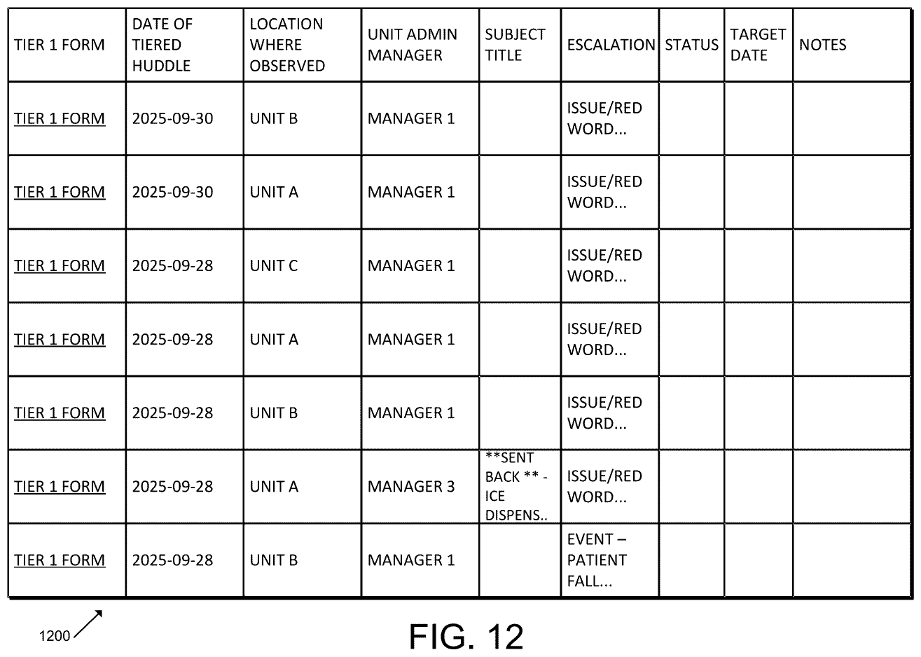

Referring now to FIG. 12, an active workboard 1200 presented to a user associated with Tier 2 (e.g., a manager) displays a set of issues. By default, the issues are sorted based on the type of escalation, with higher priority escalations (e.g., “red words”) appearing above lower priority escalations (e.g., “events”, followed by “good catches”). In the example flow of operations, the user at Tier 1 selects the top issue represented in the workboard 1200, by selecting the corresponding link in the workboard 1200. That issue is the issue reported by the user in association with the form 1100 of FIG. 11.

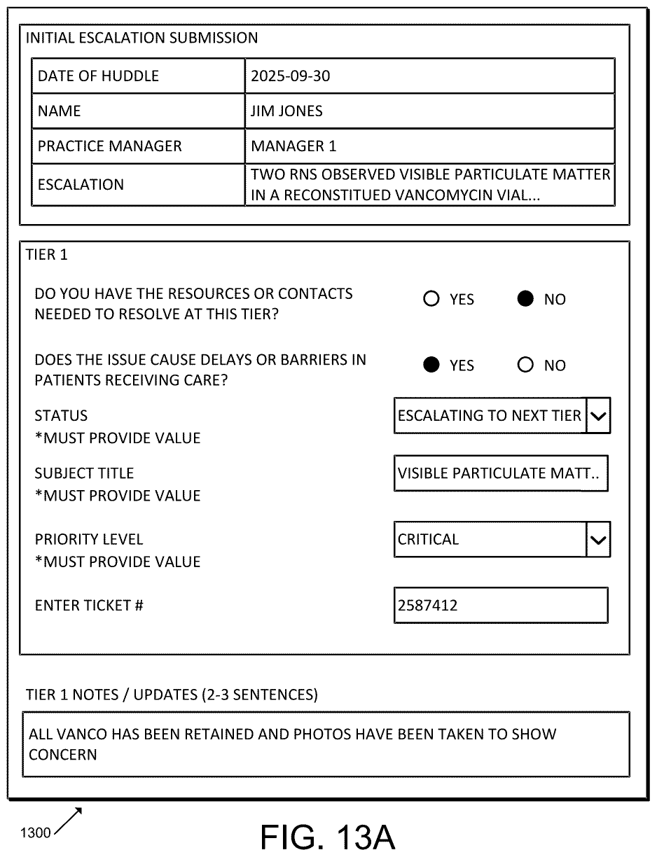

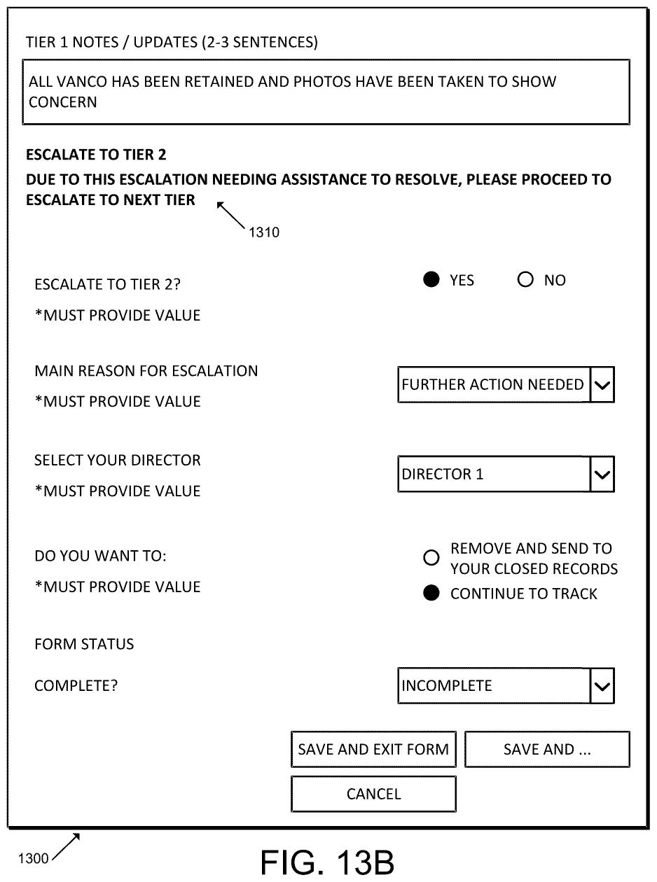

Referring now to FIG. 13A, the system 100 displays a Tier 1 version of the form 1300 associated with the selected issue. In a top section, information from the initial escalation of the issue is represented in a summarized format. Underneath the summarization of the data from the initial escalation of the issue is a section of algorithmic questions that guide whether the issue can be resolved at the present tier (i.e., Tier 1) or should be escalated to the next tier (i.e., Tier 2). Referring to FIG. 13B, based on the answers provided by the user filling out the form 1300, the system 100 has caused the form to display an instruction 1310 that the issue should be escalated to the next tier. The user selects an option to escalate and to continue to track the issue.

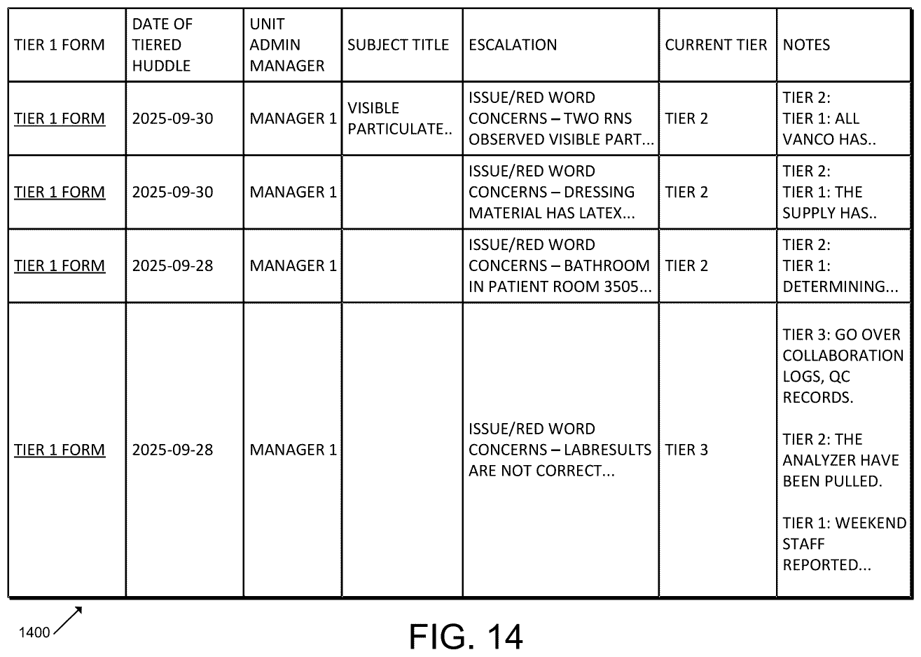

Referring now to FIG. 14, a continue to track workboard 1400 for Tier 1 shows that the issue represented at the top of the workboard 1400 has been escalated to Tier 2. For a user at Tier 2, the system 100 displays, in an active workboard similar to the active workboard 1200, a set of issues that have been escalated to Tier 2, including the present issue.

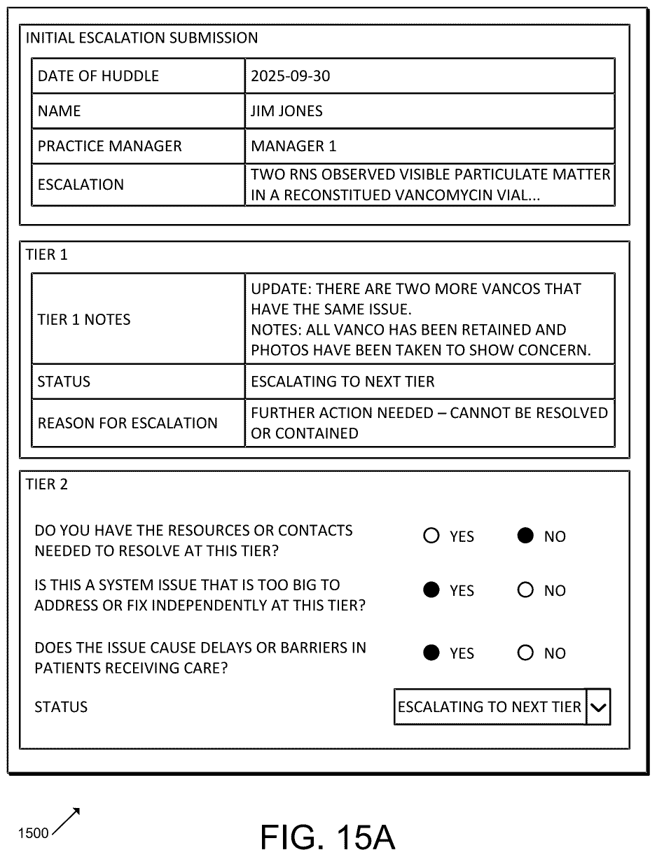

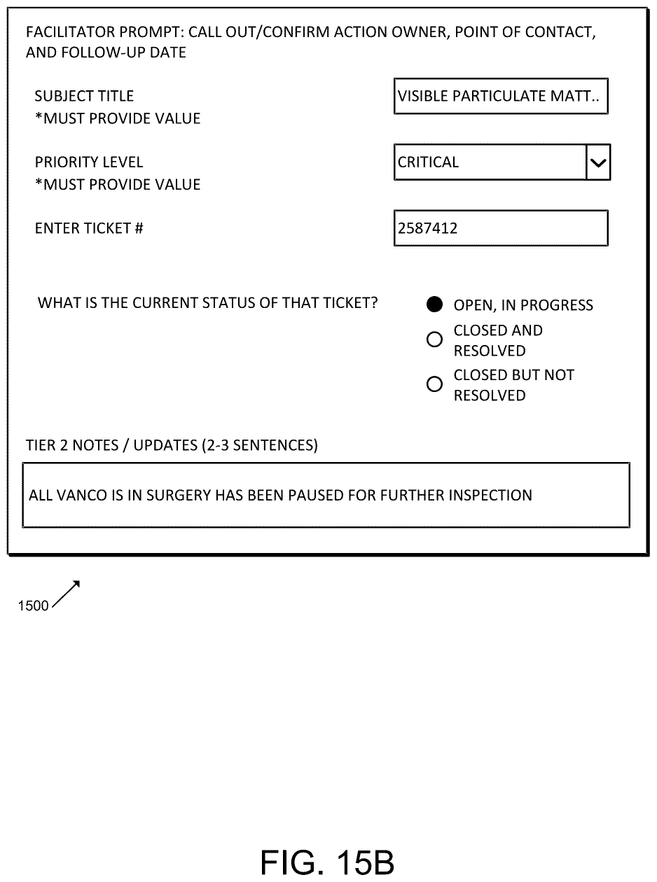

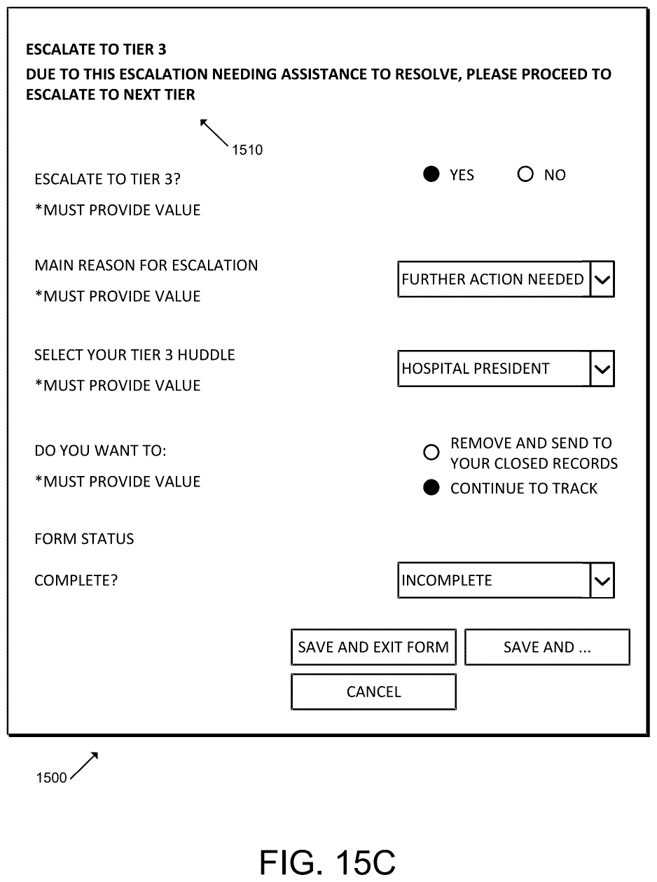

In response to selecting the present issue, the system 100 causes a Tier 2 version of the form 1500, shown in FIGS. 15A, 15B, and 15C to be presented. As shown in FIG. 15A, the form 1500 includes a section with a summarization of information provided in the initial escalation and at Tier 1, including any updates that have been subsequently entered at the corresponding tier. Below the summarization of the information provided from the earlier tier(s), a set of algorithmic questions are pre-populated based on information provided by the previous tier. If the user at Tier 2 provides different data, the system 100 stores that different data in connection with the Tier 2 version of the form 1500. As indicated in FIG. 15C, based on the information provided in response to the algorithmic questions, the system 100 has caused the form 1500 to present an instruction to escalate the issue to the next tier (i.e., Tier 3).

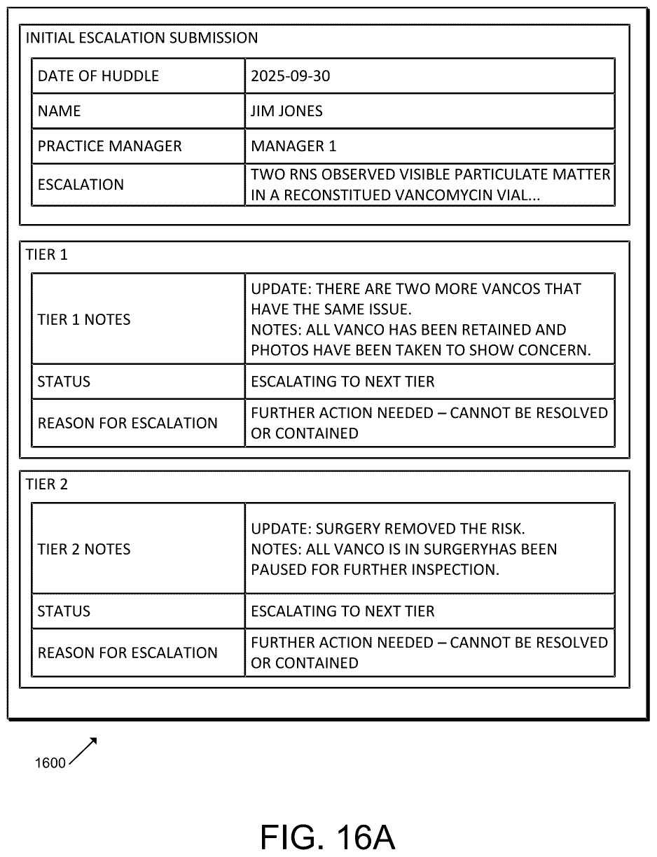

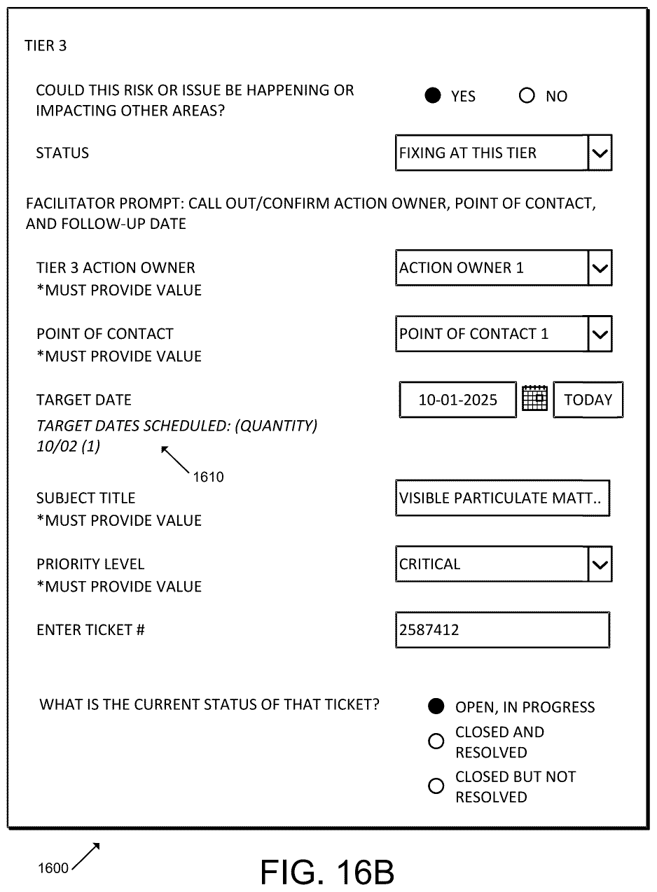

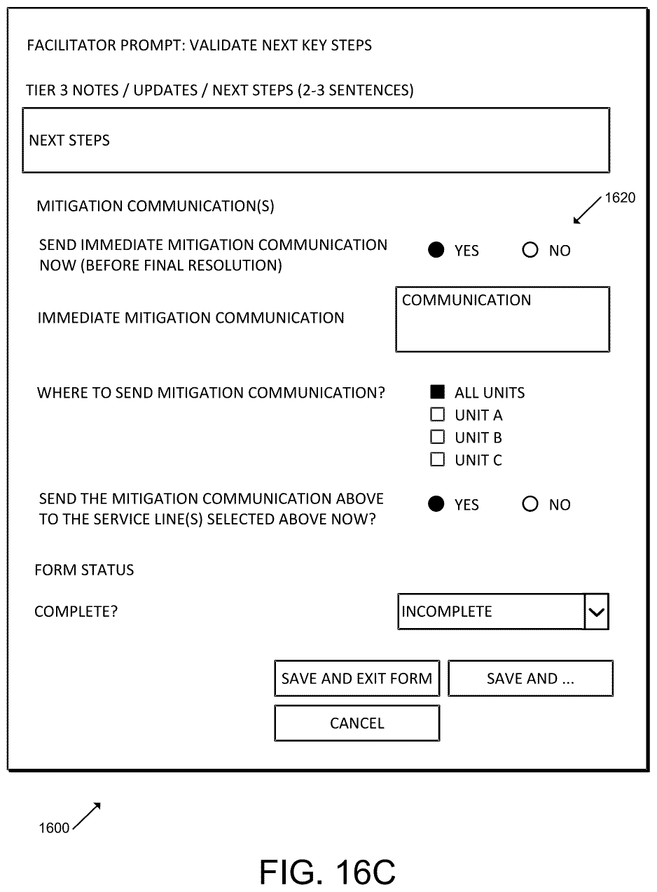

For a user at Tier 3, in response detecting that the user selected the issue in a corresponding workboard, the system 100 presents a Tier 3 version of the form 1600 associated with the issue, as shown in FIGS. 16A, 16B, and 16C. As represented in FIG. 16B, the user has indicated that the issue will be resolved at the present tier (i.e., Tier 3) and has set a target date to complete the form 1500. The system 100 compares the target date to a set of target dates of all other forms associated with the user at the present tier and displays a bottleneck message 1610 indicative of the number of other forms having the same target date. In some embodiments, the system 100 may automatically adjust the target date to a target date associated with no more than a defined number of other forms. For example, if the target date selected by the user is shared with five other forms and the maximum number of allowed forms for a target date is five, then the system 100 may change the target date to another date with fewer forms due on that date. As shown in the section 1620, represented in FIG. 16C, the user at Tier 3 enters a mitigation communication that the system 100 sends to all units.

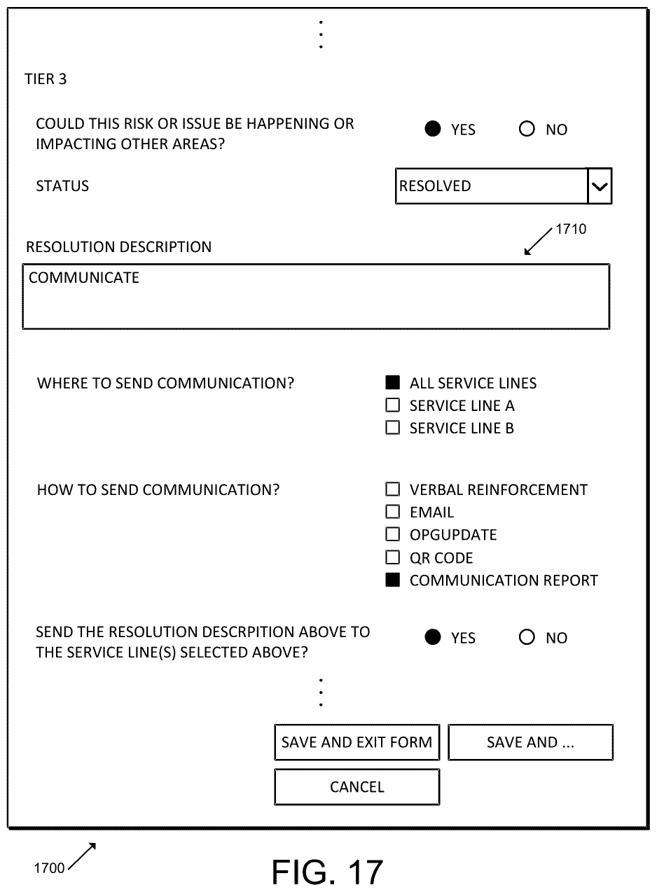

Referring now to FIG. 17, the user at Tier 3 returns to the form, by selecting it from the active workboard, and updates the form to an updated state 1700. The updated state 1700 indicates that the issue has been resolved. As indicated, the form includes a resolution communication section 1710, which appears in response to the user selecting that the issue has been resolved. The system 100 sends a communication to designated recipients (i.e., all service lines) via a selected communication method (i.e., communication report).

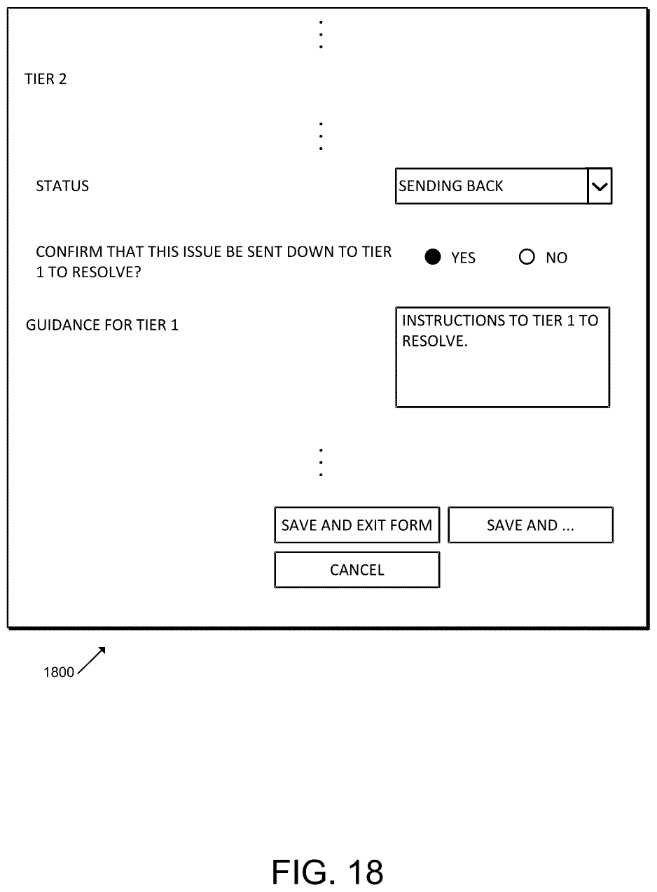

An active workboard at Tier 2 displays the issue that has been resolved at Tier 3. In response to selection of the corresponding form from the active workboard, the system 100 presents the Tier 2 version of the form in an updated state. As shown in FIG. 18, the user at Tier 2 updates the form to an updated state 1800, in which the user has selected to send the issue back to Tier 1, with a description of steps to complete to resolve the issue.

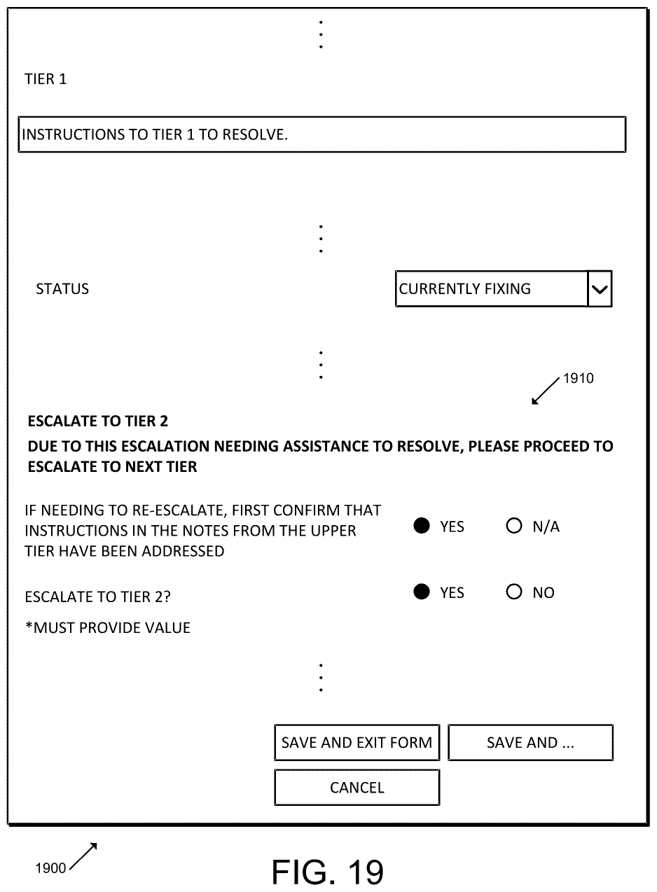

At Tier 1, a user selects the issue from a corresponding workboard, and the system 100 presents the Tier 1 version of the form as shown in FIG. 19. The user updates the Tier 1 version of the form to an updated state 1900, in which the user has indicated that the issue is currently being fixed. However, based on information provided by the user, the system 100 has caused the form to display a message 1910 indicating that the issue should be re-escalated back to Tier 2. To re-escalate the issue, the system 100 prompts the user to confirm that all instructions provided from the higher tier have been performed. Once confirmed, the system 100 re-escalates the issue back to Tier 2, at which point the issue appears in a corresponding workboard at Tier 2. The process may continue through multiple iterations of escalating and de-escalating the issue through the various tiers of the organization, with the corresponding forms at each tier representing information (e.g., updates from the lower tiers) until the issue is completely resolved.

Ownership logic uses the following rules. If the issue is escalated and not sent back and not resolved, then the current owner is the higher tier. If the ticket is escalated and resolved, the current owner is set to “Resolve at/by [that entity]”, in which “[that entity]” is replaced by an identifier of entity that resolved the issue. If the issue is escalated and sent back to a previous entity, then the current owner is set to the previous entity. If the issue is not escalated, then the current owner is set to the original entity. The system 100 evaluates the above logic for all forms associated with the issue to determine the current owner.

Core industry problems solved by the CIRC Framework include but are not limited to the following, described below, in the form of an example problem followed by a corresponding solution. CIRC enables a New Way of Exchanging Information Across Teams and Tiers.

Problem: “When I'm leading a huddle, I spend most of my time taking notes instead of solving problems. Important details and context get lost between tiers or never make it back down to the people who need them. We often run out of time before setting target follow-up dates or assigning clear action owners, so issues linger without accountability.” Solution: The invention eliminates manual note-taking and fragmented communication by capturing updates in structured, connected digital forms. Each submission circulates automatically across all tiers, preserving context and continuity. Built-in prompts ensure that every issue includes an assigned owner, a defined target date, and clear accountability for follow-up. Leaders can focus on resolving problems rather than transcribing them, ensuring every huddle drives action, closure, and communication back down the tiers.

Problem: “Trying to enter an issue into the shared document is difficult when many people are using it at the same time. Entries overlap or get deleted accidentally. There is no way to assign action owners at your tier and escalate it up to the next tier, so only one tier can be working on it at the same time.” Solution: Each user in the invention operates from an individual active form and dashboard, thereby eliminating conflicts with shared documents. Data from all forms flows automatically into the connected tier view, preventing overwrites, preserving integrity, and allowing simultaneous data entry without loss.

Problem: “When I escalate an issue, it feels like it disappears into a black hole. I never know if it was reviewed or resolved, and I rarely get feedback.” Solution: The invention provides complete, two-way transparency. Every escalation remains visible as it progresses upward, and resolution details are automatically transmitted back to the originator. This creates a continuous feedback loop, building trust between tiers.

Problem: “I have to retype the same information in multiple places, and it doesn't always match across reports.” Solution: The invention eliminates redundant data entry through interconnected forms that auto-populate shared data fields. Information entered once is instantly visible everywhere it's relevant, improving accuracy and saving time.

Problem: “When I have an escalation that I can resolve locally at my tier but still need to escalate to the upper tier for system-wide visibility, there's no way for me to continue working on it once it's escalated.” Solution: The invention enables simultaneous tier activity through a shared, connected CIRC framework. Lower tiers can continue working on their local components while the same item is escalated upward for system-level oversight. Both tiers can view each other's progress and updates in real time, ensuring complete transparency and preventing duplicate work.

Problem: “It takes longer to resolve issues because the action owners are not prepared to report out-they didn't realize they were due that day.” Solution: The invention includes automatic pre-alerts sent to each action owner the day before their assigned report-out. The alert contains a summary of their pending items and prior notes, enabling them to prepare and provide timely updates, keeping resolutions on track.

Problem: “Manually typing information is time-consuming, and our huddles move too fast. I can't capture information quickly enough to keep up with the discussion.” Solution: The invention employs a click-based input methodology that enables rapid data capture during fast-paced huddles. Users can enter information through guided selections, checkboxes, and predefined logic options rather than manual typing. All relevant data from lower tiers and previous linked forms is automatically pre-populated, ensuring speed, accuracy, and completeness without slowing the conversation.

CIRC enables a new way of ensuring appropriate escalation, return communication, and AI-guided decision-making. The following are problem and solution pairs relating to this feature.

Problem: “When I'm leading a huddle, I'm not sure whether an issue should be escalated or handled at my level.” Solution: The invention utilizes a decision-tree algorithm that automatically guides users through escalation criteria, including severity, safety impact, and urgency. Based on user responses, the system prompts escalation when specific criteria are met and recommends local resolution when they are not, ensuring consistent, timely, and data-driven decision-making.

Problem: “Sometimes I don't know what to do next. I wish the system could help me figure out the best way to handle a situation.” Solution: The invention integrates AI-driven recommendations that analyze context, prior resolutions, and user roles to suggest next steps or best practices. When uncertainty is detected, the system presents guided workflows or pre-built response options, allowing users to act confidently and consistently.

Problem: “When I determine that an issue can be resolved at the lower tier, I have no way to send it back with instructions. It depends on a manual process or verbal exchange, which delays resolution.” Solution: The invention supports bi-directional tier routing. Escalated items that can be handled locally are automatically sent back down with attached instructions or notes. These auto-populate on the originating user's form, maintaining traceability and reducing the need for rework.

Problem: “When I have an update for an issue I have already escalated, I don't have a way to send it to upper tiers. By the time it's shared, leadership is already making decisions on old information.” Solution: The invention enables real-time updates at lower tiers even after escalation. Updates entered in the original form are automatically reflected in the corresponding upper-tier record, ensuring that leaders always see the most current information.

Problem: “When I'm reviewing escalations, I can't tell when something has been updated since I last checked. It blends in with reviewed information and is easy to overlook.” Solution: The invention introduces intelligent update indicators that flag new or modified information on the workboards and within the form. Updated records are flagged with clear time stamps and highlights, ensuring leadership never overlooks recent changes.

Problem: “When I send an update, I don't know if anyone has seen or reviewed it.” Solution: The invention includes automated acknowledgment tracking. Once an upper tier reviews an update, confirmation instantly appears in the originator's form, creating visibility and reinforcing accountability across all levels.

Problem: “When an at-risk patient is identified, there's no system to automatically route that information to the right personnel or escalate oversight if preventive actions are delayed.” Solution: The invention applies its automated escalation logic to patient safety data, ensuring that high-risk patient information identified in the EMR automatically flows to the responsible tier (nurse, manager, or leadership). If an assigned preventive action is not completed or verified within its timeframe, the item turns red and automatically escalates to leadership for immediate oversight.

Problem: “Even when staff know a patient is at risk, there's inconsistency in what actions should be taken to prevent harm.” Solution: The invention extends its AI-guided recommendation engine to preventive workflows, providing role-based guidance for intervention. When a specific risk type (e.g., sepsis indicators, pressure injury) is detected, the system presents predefined evidence-based action options through a click-based interface, ensuring standardized, data-driven decision-making across all units.

Problem: “As a facilitator or active participant, I'm focused on leading the discussion and solving problems. I don't have the bandwidth to simultaneously recall all the details in the discussion. Solution: The invention uses AI-assisted logic to automatically populate required fields, such as action owner, next steps, escalation status, and target dates based on the discussion context and current form state. Before saving, the system prompts the facilitator or active participant to review and confirm the populated information. This ensures accuracy and completeness without interrupting the discussion, allowing leaders to stay focused on decision-making while the system handles documentation support.

Problem: “I receive reports from external systems showing elevated risk, but there's no clear way to turn that information into action. Risks are identified, but ownership, follow-up, and escalation are manual, inconsistent, or delayed.” Solution: The invention interfaces with external systems to ingest structured risk data and automatically generate corresponding risk items within the platform. These items are routed to the appropriate tiers for action and oversight, enabling parallel local response and leadership visibility. Mitigation actions and updates are synchronized across tiers, ensuring proactive management of risk before adverse outcomes occur.

CIRC also provides a new way of sending safety alerts and critical communications within the platform. The following are problem and solution pairs related to this feature.

Problem: “When something urgent happens, like a safety alert or a critical operational change, we can't get the message out quickly to everyone who needs it. We must leave the platform to send emails, which becomes complicated when only certain departments or hospital systems need the communication.” Solution: The invention features an integrated communication-report function that enables organization-wide or targeted alerts to be sent directly within the platform. The user selects one or more departments, service lines, or hospitals, attaches the message, and submits it. The system then distributes internal alerts to users in-platform and sends high-priority emails externally. Messages to non-platform users can be automatically sent under the hospital president's name, ensuring timely and authoritative delivery to all recipients.

Problem: “It takes too long for urgent issues to reach leadership. By the time the message gets to the right person, the problem has already escalated.” Solution: The invention provides automated, context-aware escalation for high-risk items. Issues meeting predefined thresholds are sent instantly to the next tier with complete contextual data, ensuring leadership awareness and rapid intervention.

Problem: “When a clinical risk escalates, such as a patient showing early signs of infection, we have no fast, system-integrated way to alert leadership or cross-functional teams.” Solution: The invention uses the same internal safety alert engine to send urgent notifications tied to EMR-triggered preventive risks. Once an EMR flag exceeds a defined severity threshold, the CIRC platform sends a real-time safety alert directly to relevant clinical and operational leaders within the same workflow environment, ensuring immediate visibility without relying on external email systems.

Problem: “Sometimes too many people can see sensitive information, and other times the right people can't access what they need.” Solution: The invention features role-based permissions that govern visibility and management access by tier, role, and user type. This maintains operational transparency while protecting sensitive data.

Problem: “I struggle to manage projects that require multiple teams to work at the same time. Work doesn't move in a straight line, but my project tools assume one owner at a time and break down when execution and escalation need to happen simultaneously.” Solution: The invention supports parallel project execution by allowing multiple roles and teams to work on the same project item concurrently. Execution, escalation, and oversight occur at the same time through connected forms and workboards, without forcing artificial handoffs or stopping work. The information comes to the user, instead of the user coming to the information.

Problem: “As a scribe, I have to listen to fast-moving conversations and extract critical details such as action owners, next steps, escalation decisions, and follow-up target dates. When discussions are ambiguous or incomplete, I'm forced to infer information on the fly, which increases cognitive load and creates risk that information is captured incorrectly.” Solution: The invention uses AI-assisted logic to populate required fields based on the conversation context and current form state. Before saving, the system prompts the user to confirm or adjust the populated action owner, next steps, escalation status, and target dates. This confirmation step ensures accuracy, reduces cognitive burden on the scribe, and preserves a smooth meeting flow while maintaining complete and reliable documentation.

CIRC enables a new way of maintaining visibility, compliance, and accountability across the system. Related problem and solution pairs are provided below.

Problem: “When I'm leading the huddle, I can't quickly see what I'm supposed to address today. The issues I need to follow up on are mixed with future tasks or ones I'm tracking from upper tiers.” Solution: The invention introduces logic-based workboards that automatically organize items by purpose and timing: Huddle Workboard—items requiring attention today; Follow-Up Queue—future-dated tasks; Continue-to-Track Board—ongoing escalations awaiting upper-tier action. This ensures each meeting is focused, time-efficient, and purpose-driven.

Problem: “I want to address the most critical concerns first, but manually sorting by priority slows me down and makes meetings longer. It's easy to miss high-risk issues.” Solution: The invention utilizes priority-driven sorting logic to automatically rank issues based on risk, urgency, and escalation level. High-priority items rise to the top for immediate attention, ensuring that critical problems are addressed first.

Problem: “When we assign follow-up target dates, we have no way of knowing how many follow-ups are scheduled for the same day.” Solution: The invention's bottleneck feature automatically displays the number of issues assigned for follow-up on any given day and alerts users when a specific date exceeds capacity. The system recommends redistributing follow-ups across future dates to maintain balanced workloads and efficient huddle durations.

Problem: “How do I know if departments or associates are using the platform? I need to see if they're submitting escalations and addressing escalations at their tier.” Solution: The invention captures engagement and utilization analytics. Dashboards display submission frequency, tier participation, and completion rates by department, enabling leadership to track adoption, identify areas of disengagement, and target support where needed. It also sends automatic alerts to that tier's leader if escalations remain unaddressed for a specified period, allowing for real-time intervention.

Problem: “Leadership doesn't have real-time visibility into whether preventive actions for high-risk patients are completed, overdue, or ignored.” Solution: The invention extends its color-coded compliance dashboards to clinical prevention workflows. Each preventive action automatically updates its color status across all tiers. Overdue items trigger alerts to unit leaders, ensuring immediate accountability and maintaining live system-wide visibility of all preventive actions.

Problem: “We have EMR reports of high-risk patients, but there's no visibility into whether those risks are actively being managed across units.” Solution: The invention combines EMR data with CIRC's tiered workboards, enabling managers and leaders to view both patient-level risks and the corresponding preventive actions in progress. This merges clinical data with operational accountability, enabling continuous oversight and cross-unit comparison.

Problem: “How can we create data analytics from what is being escalated? I want to see which categories are raised most often, how long they take to resolve, and whether we're escalating appropriately.” Solution: The invention generates real-time analytics and trend dashboards that visualize escalation frequency, category type, and resolution times. It identifies recurring themes and holdups, enabling proactive system improvements and data-driven decisions.

Problem: “Once an issue is marked closed, it disappears. When the same problem returns later, we must start from scratch.” Solution: The invention enables any closed issue to be instantly reactivated, retaining all historical data intact. This preserves context, eliminates duplication, and supports continuous improvement and learning.

Problem: “As a hospital leader, I need a quick daily snapshot of operations across all departments. There's no unified view showing what needs to be verbally reported out versus information for awareness only.” Solution: The invention introduces an Operational Workboard that consolidates daily report-outs from all departments into one real-time dashboard. Users enter their operational report into the submission form, and it is populated on the operational workboard. Updates are automatically populated as Verbal Report-Outs, FYI Only, or No New Updates. They are reset daily to ensure accuracy and eliminate the need for manual clearing of the workboard. The user can export the workboard to an Excel sheet if desired.

Problem: I struggle to identify which project tasks need my immediate attention. Active work, future work, and items waiting on others are mixed, which slows execution and makes it easy to miss critical actions. Solution: The invention introduces logic-based project workboards that separate near-term execution from future planning and monitoring. Items automatically appear on the appropriate workboard based on timing, readiness, and ownership, allowing users to focus on what requires action now without losing visibility into downstream work.

Problem: “I often don't realize a project is at risk until deadlines are missed. Work looks active even when progress is stalled because required input or decisions from other teams haven't occurred.” Solution: The invention continuously evaluates project activity to detect stalled work and blocked progress. When required inputs, approvals, or decisions have not occurred within defined timeframes, the system automatically flags the item and surfaces it on an at-risk or escalation workboard, enabling early intervention before delivery is impacted.

CIRC enables a new way of predicting and preventing adverse outcomes through real-time analytics and EMR integration. Related problem and solution pairs are set out below.

Problem: “Hospitals are reactive. We only recognize high-risk patients after harm occurs because there's no automated way to identify and act on emerging risks in real time. Solution: The invention integrates directly with the EMR to transform static risk reports (such as infection, sepsis, fall, or pressure injury indicators) into live, tiered preventive workboards. These workboards automatically assign and track preventive actions across all organizational levels, ensuring proactive oversight to prevent harm from occurring.

Problem: “By the time leadership sees infection data or outcome metrics, harm has already occurred.” Solution: The invention merges EMR data, CIRC's escalation logic, and color-coded compliance dashboards into a unified preventive intelligence system. When EMR thresholds are met, CIRC automatically generates a list of high-risk patients on the unit workboards within the platform to initiate action planning and resolution. It activates evidence-based preventive workflows to ensure standardized responses, prompts users to assign ownership, and verifies completion through the tiered huddle's closed-loop tracking. Leadership dashboards display real-time preventive analytics, highlighting overdue actions and emerging risk trends. This enables early intervention, sustained accountability, and continuous prevention across the enterprise, reducing hospital-acquired infections and complications.

In a data comparison between the old method (i.e., a shared Excel sheet on Microsoft Teams) and the CIRC digital platform, the magnitude of improvement the platform enables is set forth below.

### Performance Comparison (8 Months) for 400 Ambulatory Offices:

### Before—Shared Excel Sheet:

1,147 total submissions (143/month)
25% documented resolutions
18% documented communication

### After-CIRC Platform:

10,025 total submissions (1,253/month)
93% documented resolutions (median 1 day)
100% documented communication

The difference is dramatic, and the data speaks for itself. In addition to these outcome improvements, the CIRC platform also provides automated analytics that were not possible with the Excel-based method. These include: event types and issue categories, priority-level timeliness and median days to resolution, communication method tracking, department-level usage and huddle compliance, risk identification, and escalation appropriateness and algorithm alignment. While analytics themselves aren't novel, the key point is that conventional approaches (e.g., Excel) could not generate any of the above. The CIRC platform finally provides structure, and the automation that make the analytics possible.

In view of the foregoing, at a system level, CIRC may be best understood as a computing platform that generates real-time operational intelligence from information created while work is actively being carried out, rather than after-the-fact documentation. At least one of the inventive contributions lies in the machine-executed, tier-aware logic that governs changes in issue status, authority, escalation, and the automatic flow of real time information across tiers during safety huddles.

Existing healthcare systems may identify risks but typically require repeated data entry and lack an effective way to escalate issues to the appropriate level of leadership. This often results in delays, loss of context, and an emphasis on documentation instead of action. As will be appreciated from the foregoing, CIRC enables real-time, bidirectional flow of risk information across organizational tiers, supporting timely decision-making, clear ownership, and defined timelines, thereby shifting organizations away from reactive documentation to real-time execution that supports early detection of risk, leadership visibility, and corrective action before patient harm or system failure occurs.

In at least some embodiments, CIRC allows the users to address the same issue, at the same time, on different tiers, using their own independent form (workspace), while seeing real-time updates/information entries from other users connected independent forms, who are also simultaneously working on the same issue. In other words, each user may interact with a tier specific form instance that remains the controlling source for that users' actions, with the system governing how the information is propagated to other tiers without uncontrolled overwriting.

In at least some embodiments, CIRC may evaluate issue data and issue state using stored logic and algorithms. Based on that evaluation, it may: automatically execute actions (e.g., escalation or routing), and/or provide algorithm-driven guidance or recommendations to users, including escalation and/or recommendation on how to address issues that are escalated. This may allow the organization to standardize and optimize decision-making while retaining human judgment. In some embodiments this may take the form of algorithm guided decision trees and/or prompts.

In some embodiments, communication actions are executed directly within the form and/or workflow, eliminating the need for users to leave the application or perform separate manual follow-up steps. Users may initiate broad or targeted distribution of communications within a few interactions using system-defined logic and/or organizational configuration. Distribution groups may be preconfigured based on tier, role, department, facility, or organizational scope, which may allow communications to be rapidly disseminated to large groups, including leadership across multiple units or facilities. The system may support priority-aware communication, including high-priority or high-alert notifications, and may deliver messages through multiple channels, such as email or mobile device alerts, based on configured urgency and escalation rules. This may promote critical safety-related information being communicated quickly, visibly, and reliably. It should be appreciated that this communication capability may be system-managed, logic-driven, and/or integrated into issue resolution, which may promote routine, swift, and dependable dissemination of information directly from within the form, rather than relying on external tools or ad hoc manual communication.

In at least some embodiments, CIRC may intentionally constrain issue capture, escalation, and resolution inputs through structured, click-based fields (discrete fields) with limited free-text entry, rather than open-ended narrative workflows. This may ensure issue data is consistently categorized, machine-readable, and comparable across users, tiers, and time. By enforcing structured field selection aligned to predefined categories, escalation criteria, decision paths, and timing parameters, the system may promote reliable system evaluation of performance and behavior. This may include assessment of resolution timeliness, escalation patterns, category trends, and alignment with algorithmic guidance when the system is configured in a guidance mode. This may enable the system to standardize behavior, evaluate compliance and process effectiveness, and/or identify organizational risk or improvement areas, rather than treat analytics or dashboards as standalone reporting features.

In at least some implementations, the system may be configured to interface with an Electronic Medical Record (EMR) or other clinical documentation system to receive structured data related to patient status, clinical observations, or documented risk factors. Based on organization-defined criteria, rules, or thresholds, the system may identify patients who meet predefined risk conditions associated with potential complications, such as hospital-acquired infections, prior to the occurrence of an adverse event. For example, an external EMR system may generate a report or data feed identifying patients who satisfy specific risk criteria based on documented clinical inputs. The system may ingest this information and automatically generate corresponding risk items within the platform, routing them to one or more tiers for review and action. The system may enable early identification, escalation, and coordination of clinical risks before adverse outcomes occur through this integration-driven workflow. The system may support timely intervention, shared situational awareness, and accountability without replacing clinical judgment or EMR functionality by surfacing potential issues proactively and routing them through structured tier-based workflows.

What is claimed is:

1. A method for enabling efficient performance of medical services via enhanced issue tracking and resolution and huddle management, the method comprising:
receiving, by a computing system, a request to present a form associated with an issue to be resolved by a health care organization, wherein the issue affects one or more medical services provided by personnel, including nurses, of the health care organization for patients, and wherein the request is associated with a user that is associated with a corresponding tier of the health care organization;
presenting, by the computing system, a form to represent issue data indicative of the issue to be resolved by the health care organization in association with one or more huddles; and
storing, by the computing system, the issue data represented in the form, in association with a version of the form for the tier of the health care organization corresponding with the user, wherein at least a portion of the issue data is stored to be prepopulated into a different version of the form for a different tier of the health care organization to facilitate efficient resolution of the issue by the health care organization and to thereby enable efficient performance of the medical services provided by the health care organization.

2. The method of claim 1, further comprising:
determining by an issue routing system, whether the issue data warrants escalation or de-escalation of the issue; and
selectively escalating or de-escalating, by the computing system, the issue as a function of the determination of whether the issue data warrants escalation or de-escalation of the issue.

3. The method of claim 1, wherein presenting the form to represent issue data comprises presenting a form with a set of fields to each represent a corresponding portion of the issue data.

4. The method of claim 1, wherein presenting the form comprises presenting a version of the form with fields associated with the tier of the user.

5. The method of claim 1, wherein presenting the form comprises presenting a form that is associated with an existing issue associated with the health care organization.

6. The method of claim 5, further comprising retrieving data to pre-populate fields of the form as a function of issue data stored in association with at least one version of the form associated with a tier other than the tier of the user.

7. The method of claim 1, further comprising prompting the user to enter portions of the issue data as a function of workflow logic defined in association with the form.

8. The method of claim 1, further comprising presenting bottleneck data indicative of a number of other issues having a common due date with the issue associated with the form.

9. The method of claim 8, further comprising:
receiving an adjusted due date based on the presented bottleneck data; and
presenting recalculated bottleneck data as a function of the adjusted due date.

10. The method of claim 1, wherein storing the issue data represented in the form comprises overwriting prepopulated data from one or more other versions of the form associated with one or more other tiers with data entered by the user into the corresponding fields, such that the version of the form for the tier of the health care organization corresponding with the user remains independent from the one or more other versions of the form associated with the one or more other tiers, and issue data is selectively propagated between versions of forms by the computing system;
wherein presenting the form comprises presenting, to the user, communications associated with the form and designated for distribution to the user based on a distribution group; and
wherein presenting the form further comprises constraining user input to the form such that the issue data is categorized, machine-readable, and comparable to issue data in other forms.

11. The method of claim 1, further comprising:
determining, by an issue routing system, whether the issue data warrants escalation or de-escalation of the issue;
selectively escalating, by the computing system, the issue as a function of the determination of whether the issue data warrants escalation or de-escalation of the issue; and
providing, by the computing system, a notification to a target user associated with a higher tier than the tier of the user to review the issue data in a corresponding version of the form for the tier of the target user.

12. The method of claim 1, further comprising:
determining, by an issue routing system, whether the issue data warrants escalation or de-escalation of the issue;
selectively escalating, by the computing system, the issue as a function of the determination of whether the issue data warrants escalation or de-escalation of the issue; and
providing a notification to a target user associated with a lower tier than the tier of the user to review the issue data in a corresponding version of the form for the tier of the target user.

13. The method of claim 1, further comprising displaying a workboard that represents a set of issues to be resolved by the health care organization and that are associated with the user.

14. The method of claim 13, further comprising displaying a summary of issue data associated with each issue of the set of issues represented by the workboard.

15. The method of claim 13, further comprising displaying a status of each issue of the set of issues represented by the workboard.

16. A computing system for enhanced issue tracking and resolution, the computing system comprising:
at least one processor; and
at least one memory comprising a plurality of instructions stored thereon that, in response to execution by the at least one processor, causes the computing system to:
receive a request to present a form associated with an issue to be resolved by an organization, wherein the request is associated with a user that is associated with a corresponding tier of the organization;
present a form to represent issue data indicative of an issue to be resolved by the organization; and
store the issue data represented in the form, in association with a version of the form for the tier corresponding with the user, wherein at least a portion of the issue data is stored to be prepopulated into a different version of the form for a different tier of the organization.

17. The computing system of claim 16, wherein the instructions additionally cause the computing system to:
determine by an issue routing system, whether the issue data warrants escalation or de-escalation of the issue; and
selectively escalate or de-escalate the issue as a function of the determination of whether the issue data warrants escalation or de-escalation of the issue.

18. The computing system of claim 16, wherein to present the form to represent issue data comprises to present a form with a set of fields to each represent a corresponding portion of the issue data.

19. The computing system of claim 16, wherein to present the form comprises to present a version of the form with fields associated with the tier of the user.

20. The computing system of claim 16, wherein to present the form comprises to present a form that is associated with an existing issue associated with the organization.
